(19) United States
(12) Patent Application Publication
(10) Pub. No.: US 2026/0138117 A1
(43) Pub. Date: May 21, 2026

(54) ADSORPTION TECHNOLOGY

(71) Applicant: THE REGENTS OF THE UNIVERSITY OF COLORADO, A BODY CORPORATE, Denver, CO (US)

(72) Inventor: Melanie Joy, Denver, CO (US)

(73) Assignee: THE REGENTS OF THE UNIVERSITY OF COLORADO, A BODY CORPORATE, Denver, CO (US)

(21) Appl. No.: 19/116,941

(22) PCT Filed: Sep. 29, 2023
(86) PCT No.: PCT/US2023/075592

§ 371 Date: Mar. 28, 2025

Related U.S. Application Data
(60) Provisional application No. 63/411,309, filed on Sep. 29, 2022.

(51) Int. Cl. B01J 20/06; A61M 1/14; B01J 20/20; B01J 20/32

(52) U.S. Cl. CPC B01J 20/06; A61M 1/15632; B01J 20/20; B01J 20/3204; B01J 20/3236

(57) ABSTRACT

The present disclosure is related to chemical adsorption devices, systems, and methods. In particular, an inorganic ion or metabolite adsorption device includes, but is not limited to, an adsorbent substance and an adsorbing complex, the adsorbing complex being reactive to selectively adsorb an inorganic ion or metabolite from a biofluid passing over and/or through the adsorbent substance, to remove at least a portion of the inorganic ion or metabolite from the biofluid.

### CROSS-REFERENCE TO RELATED APPLICATIONS

This application claims priority to and the benefit of U.S. Provisional Patent Application Ser. No. 63/411,309 entitled “PHOSPHATE ADSORPTION TECHNOLOGY” filed on Sep. 29, 2022. The foregoing application is hereby incorporated by reference in its entirety for all purposes, including but not limited to those portions that specifically appear hereinafter, but except for any subject matter disclaimers or disavowals, and except to the extent that the incorporated material is inconsistent with the express disclosure herein, in which case the language in this disclosure shall control.

### FIELD

The present disclosure relates to chemical adsorption devices, systems, and methods. In particular, a chemical adsorption device, as disclosed herein, includes, but is not limited to, an adsorbent substance and an adsorbing complex attached to the adsorbent substance, the adsorbing complex being reactive to selectively adsorb a substance from a biofluid, or other fluid, passing over and/or through the adsorbent substance, to remove at least a portion of the substance from the biofluid/fluid and/or body storage sites.

### BACKGROUND

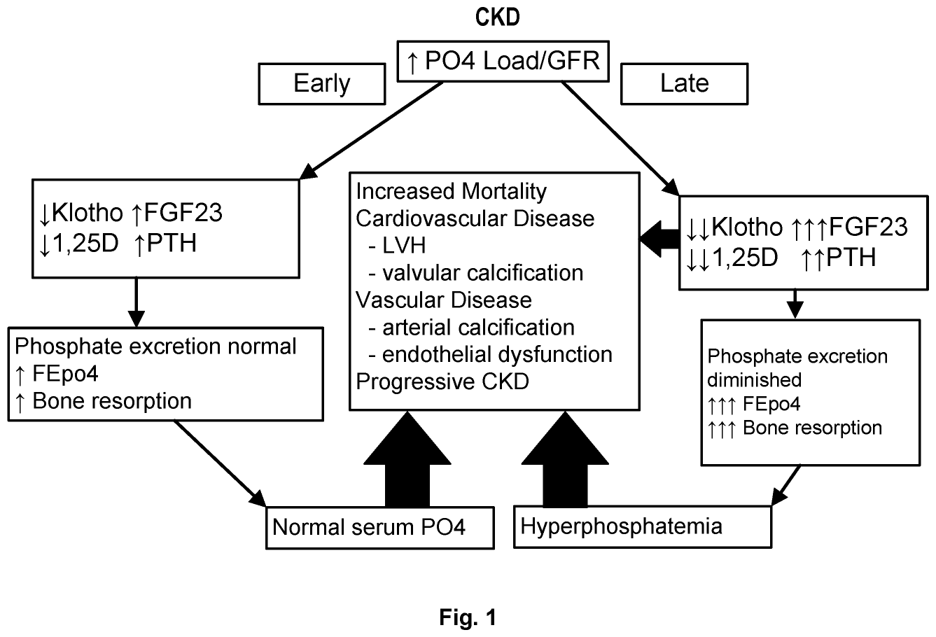

Undesirable substances are often present in biofluids and can lead to electrolyte imbalances and/or increases in total stores in the body. For example, elevated phosphate ions present in blood, for instance, can result in a medical condition known as hyperphosphatemia. See FIG. 1. With a decrease in GFR and increase in the phosphate load relative to GFR, various factors involved in mineral homeostasis are perturbed. Some changes, such as the rise in parathyroid hormone (PTH) and fibroblast growth factor 23 (FGF23), are adaptive changes that increase the fractional excretion of phosphate and maintain serum phosphate in the normal range. Other factors, such as a decline in 1,25D and Klotho, do not appear to be adaptations, per se, but contribute to the rise in PTH and FGF23, respectively. With a more severe decline in GFR (late) the efficacy of PTH and FGF23 to enhance renal excretion of phosphate diminishes due to the extensive loss of renal mass as well as resistance to their action. The bone resorptive effect of PTH also contributes to abnormal serum phosphate levels. Hyperphosphatemia, elevated PTH and FGF23 values, and low 1,25D and Klotho values all appear to independently increase the cardiovascular risk profile in CKD leading to vascular/valvular calcification, left ventricular hypertrophy (LVH), endothelial dysfunction and increased risk for cardiovascular events and mortality.

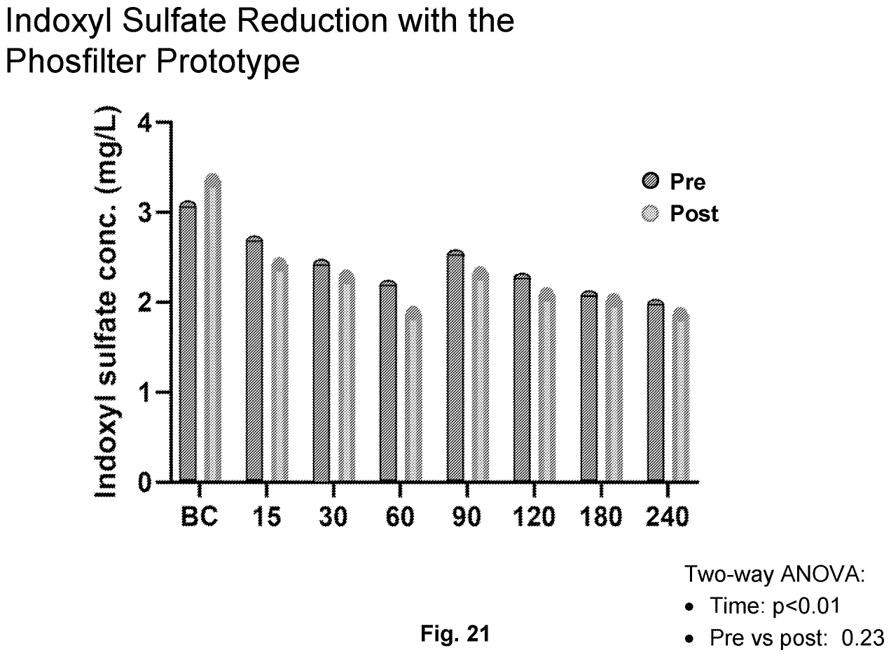

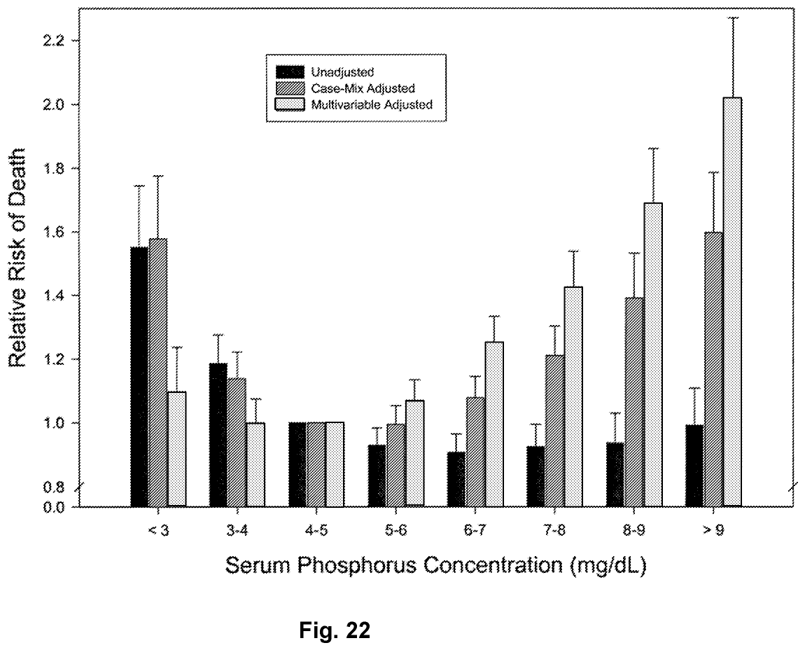

Hyperphosphatemia occurs in up to 90% of patients on hemodialysis (HD), with concentrations above the clinically acceptable threshold of 3.5 to 5.5 mg/dl in ˜50% of patients despite being prescribed low-phosphate diets and oral phosphate-binding medications. Compelling studies report clear associations between vascular calcification, endothelial dysfunction, and cardiovascular disease/mortality and high blood phosphate concentrations. Hyperphosphatemia is linked to significant morbidity and mortality rates in hemodialysis patients. Mortality rates in CKD patients increase as a function of blood phosphate levels (FIG. 22). Elevated blood phosphates (as a surrogate of net positive body burden) contribute to the 10-20 fold higher rate of death in HD patients vs. age-matched controls. Secondary to these adverse consequences of persistent hyperphosphatemia, the KDIGO guidelines recommend increasing dialytic phosphate removal and lowering blood phosphate levels toward the normal range (2.8-4.5 mg/dl). Oral phosphate-binding drugs possess finite binding capacity (40-50%), poor compliance, potential absorption of aluminum, undesirable side effects, and considerable costs. Phosphate reduction results from trials are rarely observed in clinical patients. Despite oral binder prescriptions, serum phosphate levels continue to rise. Research trial results (phosphate reductions of 1.2-2.2 mg/dL) are rarely achievable in clinical practice and serum phosphate levels continue to rise in the U.S. Prevention of a positive phosphate balance requires an increase in removal of 200-450 mg per HD session (3×/week)—a capacity that is not feasible for conventional diffusion-based 4 h HD sessions. A previous study suggested a potential for enhanced phosphate removal during HD if dialyzers had the ability to remove phosphates through the process of adsorption (extended duration, increased frequency, enhanced flows, two dialyzers in parallel, etc.).

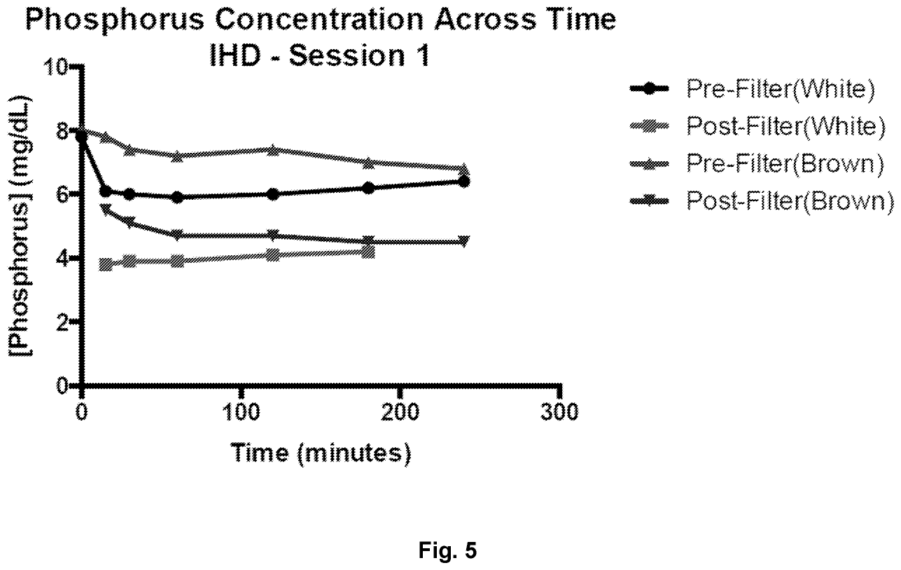

Phosphates are classified as uremic toxins, along with numerous other metabolism byproducts such as indoxyl sulfate, for example. Currently, hemodialysis is not prescribed specifically for reducing hyperphosphatemia based on achievement of some level of phosphate removed. Phosphate removal during the dialysis procedure is governed by the passive process of simple diffusion, whereby solute follows a concentration gradient from high to low concentrations in the blood and is not sufficient. See FIG. 5 showing phosphate removal over time during in vivo studies of intermittent hemodialysis in healthy canines.

Similarly, dialysis prescriptions are not currently designed for removal of uremic toxins. Many of these toxins are highly protein bound, limiting the ability of diffusion and convection, processes relevant to hemodialysis, to adequately remove them.

Phosphorus is introduced through the diet and suggested protein intake of 1.0-1.2 mg/kg/day results in a dietary phosphate load of ˜1200-1440 mg. Lowering phosphate load by decreasing protein intake is counterproductive as protein deficiency leads to increased death risk.

Currently, there are no available phosphate-selective blood filtration or adsorption devices on the market to selectively remove phosphates. Currently available HD filter membranes allow passive phosphate filtration through simple diffusion, but this process exhibits concentration-time dependent characteristics which limits amount removed over a treatment. Accordingly, there is a need for such selective filtration or adsorption devices, systems, and methods of controlling blood phosphate levels in hemodialysis patients to reduce the incidence of high phosphate-associated morbidity and mortality. Similarly, there are no available blood filtration or adsorption devices on the market to selectively remove compounds that are in the category of uremic toxins.

### SUMMARY

Disclosed is a stand-alone phosphate adsorption device, the Phosfilter. The addition of the phosfilter, to the existing diffusion-based conventional HD procedure has the potential to enhance phosphate removal and reduce net positive body burden.

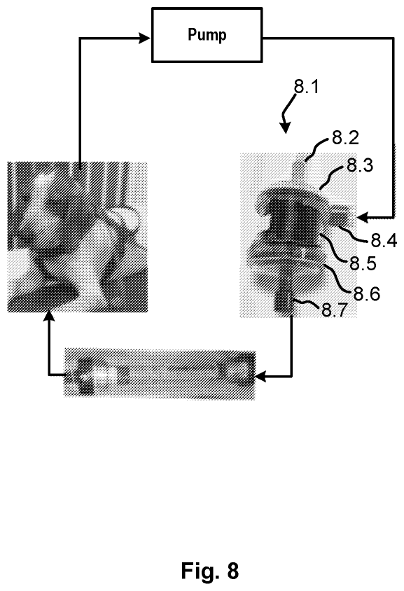

In some embodiments, the Phosfilter reduces the high phosphate-associated morbidity and mortality, diminish reliance upon out-of-clinic patient compliance and out-of-pocket costs associated with oral drugs, and creates a simplified treatment approach that enhances attainment of clinical and KDIGO benchmarks. The Phosfilter is a single use, disposable adsorbent device comprising phosphate adsorbent beads filled in housing with engineering designs incorporated to minimize damage to blood cells (FIG. 8). In some embodiments, the phosfilter comprises a plurality of different beads with different affinities. In some embodiments, the phosfilter comprises a first bead with a first affinity and a second bead with a second affinity wherein the first affinity and second affinity are different and wherein the first bead and second bead are different. In some embodiments, the phosfilter comprises a first bead with a first affinity for phosphate and a second bead with a second affinity for creatinine, calcium, magnesium, uremic toxins, and/or indoxyl sulfate wherein the first bead and second bead are different. In some embodiments, phosfilter comprises beads specific for phosphate, beads specific for creatinine, beads specific for calcium, beads specific for magnesium, beads specific for uremic toxins, beads specific for indoxyl sulfate, beads specific for lipids, or combinations thereof. Beads that are specific for a particular molecule (phosphate, creatinine, calcium, magnesium, uremic toxins, or indoxyl sulfate) have different PZC values. In some embodiments, beads that are specific for a particular molecule (phosphate, creatinine, calcium, magnesium, uremic toxins, or indoxyl sulfate) have different PZC values, bulk density, surface areas, micropore volume, meso/macro pore volume, mean pore size and/or combinations thereof. The proposed advantages of the Phosfilter device are the adsorption mechanism for phosphate removal, the potential to remove larger phosphate burdens than with diffusion-based technologies, ability to modify the device for removal of other compounds detrimental in HD patients, and use of novel chemistries and design characteristics. There are currently no existing medical devices on the market to treat hyperphosphatemia of end-stage kidney disease. In some embodiments, the adsorbent is an alginate-based bead, purigen, activated carbon, polystyrene beads coated with activated carbon, activated carbon-containing alginate bead, cellulose beads with activated carbon or combinations thereof. In some embodiments, the bead is not an activated carbon.

In some embodiments, the phosfilter is integrated into the HD circuitry and comprises iron containing activated carbon beads situated in polycarbonate housing with at least one filter at the outlet. It is accordingly an advantage of the present disclosure to provide an improved dialysis treatment device.

The removal of blood phosphates through adsorption is novel and has the potential to reduce the net positive phosphate body burden in patients on HD. Since the device is tunable, there is an opportunity for personalizing phosphate reduction prescription. Novel multicompartmental kinetic models will predict and inform about changes to phosphates in blood and tissues with Phosfilter treatment.

Disclosed are iron-containing activated carbon beads and phosfilters in buffers and blood to assess phosphate adsorption performance and safety. In some embodiments, the iron-containing activated carbon beads will enhance phosphate removal from buffers and/or blood and will have minimal impacts on other blood components. Disclosed are methods for application of the phosphate hemoadsorptive device. In some embodiments, the Phosfilter enhances in vivo phosphate clearance compared to a hemodialyzer alone in patients undergoing hemodialysis for kidney failure. The Phosfilter is integrated into the current HD session and circuitry to reduce blood and body burden of phosphates (FIG. 8). It is accordingly an object of the present disclosure to provide an improved method for hemodialysis.

Without being bound to a single theory, the device, system, and methods take advantage of the removal of phosphate from body tissues by the Phosfilter through the development of a peripheral phosphate sink to the blood. Adsorption has the propensity to result in an earlier time to reach phosphate nadirs during HD (30 min.), which could enhance phosphate removal during treatment, chipping away at the net positive body stores with each successive treatment.

The proposed advantages of the disclosed device, system, and methods over existing hemodialyzers are 1) mechanism of phosphate removal (adsorption vs. simple diffusion), 2) tunability to enable the prescribing of a phosphate adsorbing dose for the device, which is not feasible with existing hemodialyzers, 3) enhancing removal of large phosphate body burdens since adsorption has the propensity to result in an earlier time to reach phosphate nadirs than conventional dialyzers, which could enhance phosphate removal during treatment, chipping away at the net positive body stores with each successive treatment, and 4) ability to modify the device for removal of other compounds, such as uremic toxins, which are detrimental in HD patients. The disclosed device, system and methods are extracorporeal, i.e., a medical procedure or device which is outside the body. The disclosed device, system and methods can remove phosphate, creatinine, calcium, magnesium, uremic toxins, indoxyl sulfate, or combinations thereof from a patient. The disclosed device, system and methods use a micro- or nano-particle as the adsorbent substance. In some embodiments, the micro- or nano-particle comprises an activated carbon particle containing iron or iron oxide, iron oxide hydroxide, ferric oxide hydroxide, ferric hydroxide or combinations thereof. In some embodiments, the disclosed device and system have an additional filter(s) (2-5 filters) in the outlet port as a safety measure to limit any shedding of particulate of selected sizes from the device to the patient.

The present disclosure is related to chemical adsorption devices, systems, and methods. In particular, a chemical adsorption device includes, but is not limited to, an adsorbent substance and an adsorbing complex attached to the adsorbent substance, the adsorbing complex being reactive to selectively adsorb a substance from a biofluid or other fluid passing over or into the adsorbing complex, to remove at least a portion of the substance from the biofluid or fluid. In various embodiments, the adsorbent substance is a micro- or nano-particle that contains an activated carbon, that is free or bound to a substrate. In some embodiments, the nano or microparticles comprise or consist of activated carbon which is complexed (bound) with iron.

In various embodiments, the disclosure contemplates a device comprising an adsorbent substance comprising a micro- or nano-particle that contains an activated carbon, that is free or bound to a substrate. In various embodiments, the disclosure contemplates a device comprising at least one inorganic ion or metabolite-selective adsorbing complex. In one embodiment, at least one inorganic ion-selective or metabolic adsorbing complex comprises a ferric oxide hydroxide (FeOOH) group. In one embodiment, the ferric oxide hydroxide (FeOOH) group selectively adsorbs an inorganic ion of phosphate or metabolite. In one embodiment, the adsorption device comprises a hemoadsorption device, wherein said device is configured in fluidic communication with an extracorporeal blood circuit. In one embodiment, the hemoadsorption device further comprises a housing unit similar to a dialyzer or a hemoperfusion cylinder or cartridge. In one embodiment, the present disclosure contemplates a method, comprising: a) providing an adsorbent substance or material; and b) adhering an inorganic ion-selective or metabolite adsorbing complex to said adsorbent substance or material. In various embodiments, the adsorbent substance or material is a micro- or nano-particle (such as activated carbon, phosphorus sheet, or cylindrical roll), and the inorganic ion-selective or metabolite adsorbing complex is adhered to a surface of the micro- or nano-particle. In one embodiment, the adhering said inorganic ion-selective or metabolite adsorbing complex comprises the steps of: a) activating a micro- or nano-particle; b) loading said micro- or nano-particle with said at least one inorganic ion-selective or metabolite adsorbing complex; and c) filling said loaded micro- or nano-particle to a cylinder, tube, or cartridge to adhere said at least one inorganic ion-selective or metabolite adsorbing complex. In one embodiment, the method further comprises the step of adding a mesh overlay over a polymer sheet. In one embodiment, the method further comprises the step of forming said absorbent substance into a cylindrical roll. In some embodiments, the micro- or nano-particle is an iron-based adsorbent in the presence of iron-containing oral phosphate binders. In one embodiment, the method further comprises inserting said cylindrical roll within a housing. In various embodiments, the adsorbent substance or material comprises a micro- or nano-particle that may contain an activated carbon, and the step of adhering said inorganic ion-selective or metabolite adsorbing complex comprises loading at least one inorganic ion-selective or metabolite adsorbing complex to the micro- or nano-particle that may contain an activated carbon. In an embodiment, the method further comprises the step of inserting said micro- or nano-particles within a cylindrical housing.

In one embodiment, the method contemplates a method, comprising: a) providing; i) a hemoadsorptive device comprising an adsorbent substance or material comprising at least one inorganic ion-selective or metabolite adsorbing complex; and ii) a fluid comprising at least one inorganic ion or metabolite; and b) contacting said fluid with said hemoadsorptive device whereby said at least one inorganic ion or metabolite is selectively removed from said fluid. In one embodiment, the inorganic ion is phosphate. In one embodiment, the fluid is a biofluid. In one embodiment, the biofluid is blood. In one embodiment, the fluid is dialysate. In one embodiment, the adsorptive device is a hemoadsorptive device. In one embodiment, at least one inorganic ion is selectively removed from said biofluid without substantially affecting the concentration of other blood chemistry compounds. In various embodiments, the adsorbent substance or material is a micro- or nano-particle. In one embodiment, the micro- or nano-particle comprises a cured silicone gel resin. In one embodiment, the inorganic ion-selective adsorbing complex comprises a ferric oxide hydroxide group and an activated carbon.

In another embodiment, the metabolite is a uremic toxin, indoxyl sulfate for example. In one embodiment, the fluid is a biofluid. In one embodiment, the biofluid is blood. In one embodiment, the fluid is dialysate. In one embodiment, the adsorptive device is a hemoadsorptive device. In one embodiment, at least one metabolite is selectively removed from said biofluid without substantially affecting the concentration of other blood chemistry compounds, outside of uremic toxin metabolites. In one embodiment, at least one uremic toxin metabolite is selectively removed from said biofluid without substantially affecting the concentration of other blood chemistry compounds. In various embodiments, the adsorbent substance or material is a micro- or nano-particle. In one embodiment, the micro- or nano-particle comprises a cured silicone gel resin. In one embodiment, the metabolite adsorbing complex comprises a ferric oxide hydroxide group or an activated carbon.

In one embodiment, the present disclosure contemplates a method, comprising: a) providing; i) an adsorbing device comprising an adsorbent substance adhered to at least one inorganic ion-selective or metabolite adsorption complex; ii) a container comprising a first fluid at a first inorganic ion or metabolite concentration; iii) an extracorporeal filtration circuit in fluidic communication with said container; b) positioning said adsorbing device within said extracorporeal filtration circuit; c) contacting said fluid with said adsorbing device; and d) selectively removing said inorganic ion or metabolite from said first fluid to create a second fluid at a second inorganic ion or metabolite concentration. In various embodiments, the adsorbent substance is a micro- or nano-particle. In various embodiments, the container is a tube or cylinder. In one embodiment, the first inorganic ion or metabolite concentration is higher than said second inorganic ion concentration. In one embodiment, the extracorporeal filtration circuit comprises a housing similar to a dialyzer or a hemoperfusion cylinder or cartridge. In one embodiment, the housing similar to a dialyzer or a hemoperfusion cylinder or cartridge comprises a hemofilter or hemoadsorber. In one embodiment, the extracorporeal filtration circuit is an extracorporeal blood circuit. In one embodiment, the first fluid is a biofluid. In one embodiment, the biofluid is blood.

In one embodiment, the present disclosure contemplates an adsorbent system comprising: a) an adsorbent device comprising an adsorbent substance adhered to an inorganic ion-selective or metabolite adsorbing complex, said adsorbing complex being reactive to selectively adsorb an inorganic ion or metabolite from a fluid; and b) a housing disposed about said adsorbent device. In various embodiments, the adsorbent substance is a micro- or nano-particle. In one embodiment, the system is adapted to selectively adsorb at least one inorganic ion or metabolite from said fluid. In one embodiment, the fluid is a biofluid. In one embodiment, the biofluid is blood. In one embodiment, the fluid is dialysate. In one embodiment, the system is adapted for filtering or adsorbing at least one inorganic ion or metabolite from said blood without altering the concentration of other physiologic blood chemistry compounds. In one embodiment, at least one inorganic ion is phosphate. In another embodiment, at least one metabolite is a uremic toxin such as indoxyl sulfate (indole grouping), amine, amino acid, d-amino acid, and/or dicarboxylic acid. See Table 3 for additional uremic toxins.

TABLE 3
Classification of uremic toxins according physical and chemical characteristics.

| Water-soluble (<500 Da) | Middle molecule (>500 Da) | Protein-bound |
|---|---|---|
| n = 44 | n = 30 | n = 31 |
| Ribonucleoside group | Protein group | Protein group |
| 1-methyladenosine (281 Da) | Adiponectin (28,000 Da) | Angiogenin (14,400 Da) |
| N2,N2-dimethylguanosine | Basic fibroblast growth factor | Insulin-like growth factor 1 |
| (311 Da) | (BFGF) (24,000 Da) | (IGF-1) (7,650 Da) |
| N4-acetylcytidine (285 Da) | Complement factor D | Leptin (16,000 Da) |
| | (26,750 Da) | |
| Xanthosine (284 Da) | Peptide group | Osteocalcin (5800 Da) |
| 1-methylguanosine (297 Da) | Guanylin (1,516 Da) | Retinol binding protein (RBP) |
| | | (21,200 Da) |
| Inosine (268 Da) | Uroguanylin (1,668 Da) | Vascular endothelial growth |
| | | factor (VEGF) (34,250 Da) |
| 1-methylinosine (282 Da) | Vasoactive intestinal peptide | α1-acid glycoprotein (43,000) |
| | (VIP) (3,325 Da) | |
| N6-methyladenosine (281 Da) | Adrenomedullin (5,729 Da) | Hippurate group |
| N6- | Atrial natriuretic peptide | Hippuric acid (total) (179 Da) |
| threonylcarbamoyladenosine | (ANP) (3,080 Da) | |
| (378 Da) | | |
| Pseudouridine (244 Da) | Calcitonin gene-related | AGE group |
| | peptide (CGRP) (3,789 Da) | |
| Amine group | Methionine-enkephalin | Pentosidine (378 Da) |
| | (555 Da) | |
| S-adenosylhomocysteine | Motiline (2,699 Da) | 3-deoxyglucosone (162 Da) |
| (384 Da) | | |
| Monomethylamine (31 Da) | Cholecystokinin (3,866 Da) | Fructoselysine (308 Da) |
| Dimethylamine (45 Da) | Clara cell protein (CC16) | Glyoxal (58 Da) |
| | (15,800 Da) | |
| Ethylamine (45 Da) | Cystatin C (13,300 Da) | Methylglyoxal (72 Da) |
| Trimethylamine (59 Da) | Hyaluronic acid (Hyaluronan) | N(6)-Carboxymethyllysine |
| | (25,000 Da) | (CML) (204 Da) |
| Trimethylamine-N-oxide | Neuropeptide Y (4,272 Da) | Catecholamine group |
| (75 Da) | | |
| RCO group | Resistin (12500 Da) | Dihydroxyphenylalanine (PB- |
| | | DOPA) (197 Da) |
| 2-heptenal (112 Da) | Vasopressin (ADH) | Amino acid group |
| | (1,084 Da) | |
| 2-hexenal (98 Da) | Endothelin (4,283 Da) | Homocysteine (135 Da) |
| Nonanal (142 Da) | Degranulation inhibiting | Kinurenine (208 Da) |
| | Protein I (14,100 Da) | |
| 2-nonenal (140 Da) | Delta-sleep inducing Peptide | Kynurenic acid (189 Da) |
| | (848 Da) | |
| 2-octenal (126 Da) | Parathyroid hormone | Indole group |
| | (9,225 Da) | |
| 4-decenal (154 Da) | Substance P (1,348 Da) | Indican (295 Da) |
| Heptanal (114 Da) | β-2-Microglobulin | Indole-3-acetic acid (175 Da) |
| | (11,818 Da) | |
| Decanal (156 Da) | β-endorphin (3,465 Da) | Indoxyl sulfate (212 Da) |
| Hexanal (100 Da) | λ-Ig light chain (25,000 Da) | Indoxyl-β-D-glucoronide |
| | | (309 Da) |
| Amino acid group | Cytokine group | Melatonin (126 Da) |
| Dimethylglycine (103 Da) | Interleukin-18 (20,000 Da) | Quinolinic acid (167 Da) |
| 4-HO-decenal (170 Da) | Interleukin-1β (32,000 Da) | Cytokine group |
| 4-HO-hexenal (114 Da) | Interleukin-6 (24,500 Da) | Interleukin-10 (18,000 Da) |
| 4-HO-nonenal (156 Da) | Tumor necrosis factor alpha | Phenol group |
| | (TNF) (26,000 Da) | |
| 4-HO-octenal (142 Da) | | Phenol (94 Da) |
| Peptide group | | p-Cresylsulfate (187 Da) |
| β-lipotropin (461 Da) | | Polyamine group |
| Nicotinamide group | | Putrescine (88 Da) |
| 4-pyridone-3-carboxamide-1- | | Group (other) |
| β-D-ribonucleoside (272 Da) | | |
| N-methyl-2-pyridone-5- | | 3-carboxy-4-methyl-5-propyl- |
| carboxamide (152 Da) | | 2-furanpropanoic acid |
| | | (CMPF) (240 Da) |
| N-methyl-4-pyridone-3- | | Phenylacetic acid (136 Da) |
| carboxamide (152 Da) | | |
| Nicotinamide (122 Da) | | |
| Purine group | | |
| 8-hydroxy-2^{l}-deoxyguanosine | | |
| (283 Da) | | |
| Cytidine (234 Da) | | |
| Hypoxanthine (136 Da) | | |
| Neopterin (253 Da) | | |
| Uric acid (168 Da) | | |
| Xanthine (152 Da) | | |
| Polyol group | | |
| Arab(in)itol (152 Da) | | |
| Mannitol (182 Da) | | |
| Erythritol (122 Da) | | |
| Myoinositol (180 Da) | | |
| Sorbitol (182 Da) | | |
| Threitol (122 Da) | | |
| Guanidine group | | |
| Argininic acid (175 Da) | | |
| Asymetric dimethylarginine | | |
| (ADMA) (202 Da) | | |
| Creatine (131 Da) | | |
| Creatinine (113 Da) | | |
| Guanidine (59 Da) | | |
| Guanidinosuccinic acid | | |
| (175 Da) | | |
| Methylguanidine (73 Da) | | |
| Symmetric dimethylarginine | | |
| (SDMA) (202 Da) | | |
| Taurocyamine (174 Da) | | |
| α-keto-δ-Guanidinovaleric | | |
| acid (173 Da) | | |
| α-N-acetylarginine (216 Da) | | |
| β-guanidinopropionic acid | | |
| (131 Da) | | |
| γ-guanidinobutyric acid | | |
| (145 Da) | | |
| Aldehyde group | | |
| Malondialdehyde | | |
| (MDA) (71 Da) | | |
| Pyrimidine group | | |
| Orotic acid (174 Da) | | |
| Orotidine (288 Da) | | |
| Uridine (244 Da) | | |
| Group (other) | | |
| Oxalate (90 Da) | | |
| Phenylacetylglutamine | | |
| (264 Da) | | |
| Urea (60 Da) | | |

Uremic toxins are compounds that are usually filtered and excreted by the kidneys. Uremic toxins can be subdivided into three major groups based upon their chemical and physical characteristics, essentially because of the role of those characteristics in removal by dialysis strategies: (a) Small, water-soluble compounds with no or minimal protein binding, such as uric acid, TMAO, ADMA; (b) Middle molecule uremic toxins, such as TNFα; (c) Protein bound compounds, such as the indoles and phenols.

In one embodiment, the housing is similar to a dialyzer or a hemofiltration cylinder or cartridge or comprises a dialyzer or a hemoperfusion cylinder or cartridge. In one embodiment, the inorganic ion selective adsorbing complex comprises a ferric oxide hydroxide (FeOOH) group and contains an activated carbon.

In one embodiment, the present disclosure contemplates an adsorption device for reducing a level of at least one inorganic ion or metabolite in blood, the device comprising: an adsorbent substance; and an adsorbing complex attached at least partially to the adsorbent substance, the adsorbing complex being reactive to selectively adsorb the at least one inorganic ion or metabolite from blood as it contacts the micro- or nano-particle to remove the at least one inorganic ion or metabolite from the blood. In various embodiments, the adsorbent substance is a micro- or nano-particle. In one embodiment, the adsorption device is adapted for filtering at least one inorganic ion or metabolite from the blood without adversely altering the blood chemistry. In one embodiment, the adsorbing complex comprises ferric oxide hydroxide (FeOOH) and contains activated carbon. In one embodiment, the adsorption device comprises a hemoadsorption device adapted to adsorb at least one inorganic ion or metabolite from blood in an extracorporeal blood circuit. In one embodiment, the micro- or nano-particle is affixed to a cured silicone gel resin. In various embodiments, the adsorbent substance is a micro- or nano-particle that contains an activated carbon, that is free or bound to a substrate. In one embodiment, the adsorbing complex is reactive to selectively adsorb at least one inorganic ion or metabolite from dialysate flowing through a dialyzer or a hemoperfusion cylinder or cartridge.

In one embodiment, the present disclosure contemplates an adsorption device for reducing a level of uremic toxin metabolites in blood, the device comprising: an adsorbent substance; and an adsorbing complex attached at least partially to the adsorbent substance, the adsorbing complex being reactive to selectively adsorb metabolites from blood as it contacts the micro- or nano-particle to remove metabolites from the blood. In various embodiments, the adsorbent substance is a micro- or nano-particle. In one embodiment, the adsorption device is adapted for filtering metabolites from the blood without adversely altering the blood chemistry. In one embodiment, the adsorbing complex comprises ferric oxide hydroxide (FeOOH) and contains activated charcoal. In one embodiment, the adsorption device comprises a hemoadsorption device adapted to adsorb metabolites from blood in an extracorporeal blood circuit. In various embodiments, the adsorbent substance is a micro- or nano-particle that contains an activated carbon, that is free or bound to a substrate. In one embodiment, the adsorbing complex is reactive to selectively adsorb metabolites from dialysate flowing through a dialyzer or a hemoperfusion cylinder or cartridge.

In one embodiment, the present disclosure contemplates a method, comprising: a) providing an adsorption device; b) providing an adsorbent substance; and c) adhering an adsorbing complex to the adsorbent substance, the adsorbing complex being reactive to selectively adsorb a substance from an aqueous solution. In various embodiments, the adsorbent substance is a micro- or nano-particle, and the adsorbing complex is adhered to a surface of the micro- or nano-particle. In one embodiment, the adhering of an adsorbing complex to the surface of the micro- or nano-particle may include, but is not limited to the sub-steps of, loading the micro- or nano-particle with ferric oxide hydroxide (FeOOH) and contains an activated charcoal. In one embodiment, the micro- or nano-particles are loaded with ferric oxide hydroxide (FeOOH) and contains an activated charcoal.

In one embodiment, the present disclosure contemplates a method, comprising: a) providing; i) a hemofiltration device comprising a filter at least partially constructed of an adsorbent substance, wherein an adsorbing complex has been adhered to the adsorbent substance and ii) a container comprising blood and a toxic ion or metabolite; and b) interfacing the container with the hemofiltration device whereby the toxic ion or metabolite is selectively removed from the blood. In various embodiments, the adsorbent substance is a micro- or nano-particle, and the adsorbing complex is adhered to a surface of the micro- or nano-particle. In various embodiments, the adsorbent substance is a micro- or nano-particle. In one embodiment, the container is a tube, cylinder, or cartridge. In one embodiment, the toxic ion comprises at least one inorganic ion or metabolite. In another embodiment, the metabolite comprises a uremic toxin such as indoxyl sulfate. In one embodiment, the selective removal of the toxic ion or metabolite does not substantially affect other physiologic blood chemistry compounds. In one embodiment, the adsorbing complex is adhered to the surface of the micro- or nano-particle via a chemistry process know to those of skill in the art. See e.g. U.S. Patent publication no. 2019/0046567, U.S. U.S. Pat. No. 9,278,170 and International publication no. WO 2018/170320 each of which are incorporated by reference in their entirety. In one embodiment, the adsorbing complex comprises ferric oxide hydroxide (FeOOH) and may contain an activated charcoal.

In one embodiment, the present disclosure contemplates a method, comprising: a) providing: i) an adsorbing device comprising an adsorbent substance and an adsorbing complex at least partially attached to the adsorbent substance; ii) an extracorporeal blood circuit in fluidic communication with the adsorbing device; and iii) a container comprising blood flowing through the extracorporeal blood circuit; b) positioning the adsorbing device in an extracorporeal blood circuit; and c) interfacing the blood with the adsorbent device for selective removal of at least one inorganic ion or metabolite. In various embodiments, the adsorbent substance is a micro- or nano-particle, and the adsorbing complex is adhered to a surface of the micro- or nano-particle. In various embodiments, the adsorbent substance is a micro- or nano-particle. In some embodiments, the device can be used in other extracorporeal circuits, such as a circuit without a dialyzer. In some embodiments, the device can be used in a hemoperfusion circuit.

In one embodiment, the adsorbing device is placed within a dialysis circuit. In one embodiment, the adsorbing device is formed integral with a dialyzer circuit. In one embodiment, the dialysis circuit comprises a hemoadsorptive device. In some embodiments, the phosfilter and dialyzer are connected through connection tubing. In some embodiments, the phosfilter is hemocompatible. FeO(OH) has high affinity to phosphates, but it is not hemocompatible. Iron oxide/hydroxide on activated carbons shows good affinity to phosphates, its affinity is controllable, is hemocompatible, and the method is reproducible. In some embodiments, the phosfilter is used in hemoperfusion. In some embodiments, there is an additional reduction in serum phosphate concentrations of 1 mg/dL in HD plus Phosfilter combination vs. HD treatment alone.

In one embodiment, the present disclosure contemplates an adsorbent system comprising: a) an adsorbent device comprising an adsorbent substance and an adsorbing complex at least partially attached to the adsorbent substance, the adsorbing complex being reactive to selectively adsorb a substance from an aqueous solution; and b) a housing disposed about the adsorbent device. In various embodiments, the adsorbent substance is a micro- or nano-particle, and the adsorbing complex is adhered to a surface of the micro- or nano-particle. In some embodiments, the adsorbing compound is inside the carbon particles i.e., not on the surface. In some embodiments, the adsorbing compound is inside the carbon particles and on the surface of the micro- or nano-particle. In various embodiments, the adsorbent substance is a micro- or nano-particle. In one embodiment, the system is adapted to selectively adsorb at least one inorganic ion or metabolite from the aqueous solution. In one embodiment, the system is adapted for adsorbing at least one inorganic ion or metabolite from blood without altering the physiologic chemistry of the blood. In one embodiment, the housing is further similar to a dialyzer or hemofiltration cartridge. In one embodiment, the adsorbing complex is adhered to the surface of the micro- or nano-particle via a chemistry process know to those of skill in the art. In one embodiment, the adsorbing complex comprises ferric oxide hydroxide (FeOOH) and contains an activated carbon. In various embodiments, the adsorbent substance comprises a micro- or nano-particle that may contain an activated carbon, and the step of adhering said inorganic ion-selective or metabolite adsorbing complex comprises loading at least one inorganic ion-selective or metabolite adsorbing complex to the micro- or nano-particle that may contain an activated carbon. In an embodiment, the method further comprises the step of inserting said micro- or nano-particles within a cylindrical housing.

In one embodiment, the adsorption system is adapted to filter at least one inorganic ion or metabolite from dialysate in an extracorporeal blood circuit.

In one embodiment, the present disclosure contemplates an adsorption device comprising an adsorbent substance and an adsorbing complex at least partially attached to the adsorbent substance, the adsorbing complex being reactive to selectively adsorb a substance from a liquid passing over or into the adsorbent substance to remove at least a portion of the substance from the liquid. In various embodiments, the adsorbent substance is a micro- or nano-particle, and the adsorbing complex is adhered to a surface of the micro- or nano-particle. In various embodiments, the adsorbent substance is a micro- or nano-particle.

In one embodiment, the adsorption device is adapted for adsorbing at least one inorganic ion or metabolite from the blood without altering physiologic chemistry of the blood. In one embodiment, the adsorbing complex comprises ferric oxide hydroxide (FeOOH) and contains an activated charcoal. In one embodiment, the adsorption device further comprises a hemoadsorption device adapted to filter at least one inorganic ion or metabolite from blood in an extracorporeal blood circuit.

In one embodiment, the micro- or nano-particle is adhered to a cured silicone gel resin.

In one embodiment, the adsorbing complex is reactive to selectively adsorb at least one inorganic ion or metabolite from dialysate flowing through a dialyzer.

In some embodiments, disclosed is a device for selective inorganic ion or metabolite removal in HD patients, patients that are CKD (non-dialysis dependent), patients that have cancer, autoimmune disease, sepsis, AKI (acute kidney injury), inflammation, infections, intoxications, liver damage, or brain diseases. In some embodiments, hypercalcemia is managed with the device. In some embodiments, the device is single use, disposable, can be integrated into the hemodialysis circuitry configuration, consists of a at least one inorganic ion or metabolite adsorbent synthetic activated carbon particles containing iron situated in polycarbonate housing with a filter at the outlet and combinations thereof. In some embodiments, there are two filters at the outlet port. The second (or more) filters do not cause pressure build up or clotting. In some embodiments, there are one to five filters at the outlet port. In some embodiments, iron is the iron oxide, iron oxide hydroxide, zerovalent iron, ferrous hydroxide, ferrous sulfate, ferrous chloride. In some embodiments, the iron-containing activated carbon particles, are prepared by taking ammonium ferric sulfate and activated carbon as raw materials and performing a high-temperature heat treatment method. In some embodiments, the iron-containing active carbon particles serve as cores, and another compound such as arachidonic acid is grafted outside the cores. For more detail see the discussion below regarding creation of activated carbon particles containing iron.

In some embodiments, the device is tunable, i.e., there is a potential for personalizing inorganic ion or metabolite reduction prescription for treatment with the device. In some embodiments, the device is not a hemodialysis filter membrane, and does not allow passive inorganic ion or metabolite filtration through diffusion. The device, system, and methods do not use inorganic ion or metabolite binders and inhibitors of intestinal inorganic ion or metabolite transporters. In some embodiments, the disclosed device comprises blood at least one inorganic ion or metabolite absorbed onto specialized carbon particles to enable high-capacity binding. In some embodiments, the device comprises adsorptive carbon beads capable of selectively removing at least one inorganic ion or metabolite with little to no effect on the concentrations of other blood electrolytes or trace metals,

In some embodiments, disclosed is a method for the removal of inorganic ion or metabolite in the blood through the process of adsorption. In some embodiments, the method reduces the net positive phosphate body burden in patients on HD. In some embodiments, the disclosed method 1) improves blood phosphate control, creatinine control, calcium control, magnesium control, uremic toxins control, indoxyl sulfate control, or combinations thereof, 2) reduces systemic/tissue phosphate burden, 3) reduces morbidity and mortality, 4) provides a simplistic and safe treatment for phosphate reduction in patients on HD and 5) combinations thereof. In some embodiments, the disclosed method adsorbs blood phosphates onto specialized carbon particles to enable high-capacity binding. In some embodiments, the method comprises using adsorptive carbon beads to selectively remove phosphate ions with little to no effect on the concentrations of other blood electrolytes or trace metals. Without being bound to a single theory, the device, system, and methods take advantage of unique phosphate selective adsorptivity to provide significant reduction in body phosphates beyond what is offered by dialysis alone or in combination with controlled diet or phosphate binding drugs. Without being bound to a single theory, the device, system, and methods take advantage of unique uremic toxin selective adsorptivity to provide significant reduction in body uremic toxins beyond what is offered by dialysis alone or in combination with controlled diet or uremic toxin binding drugs. In some embodiments, the method comprises pumping blood through the device at typical rates (300-500 mL/min) prescribed during HD (which are known to those of skill in the art) and returned to the patient, requiring limited extracorporeal blood volume (100-300 mL).

In some embodiments, disclosed is hemoadsorbent device that can be incorporated into the conventional HD circuitry to reduce the blood and body burden of phosphate, creatinine, calcium, magnesium, uremic toxins, indoxyl sulfate, or combinations thereof for HD patients, patients with chronic kidney disease and/or a patient that is uremic. In some embodiments, the patients have tumor lysis syndrome of cancer and are treated by the removal of calcium, and/or magnesium from their blood.

In some embodiments, disclosed is a method for improved phosphate and/or uremic toxin management over currently available approaches including dialysis (diffusion), dietary restrictions, and oral medications (phosphate binding drugs and intestinal transport inhibitors).

In some embodiments, disclosed is a method to reduce the incidence of high phosphate-associated morbidity and mortality, high uremic toxin morbidity and mortality, diminish reliance upon out-of-clinic patient compliance and out-of-pocket costs associated with oral drugs and create a simplified treatment approach for patients that enhances attainment of clinical benchmarks.

In some embodiments, disclosed is an adsorption device for removing beta amyloid from deep tissue (brain) through adsorption from blood. In some embodiments, the Phosfilter is used alone or in combination with diet and/or phosphate binders.

In some embodiments, the methods and device have no or limited particulate shedding. The repeated flexion and compression of pump segments by the rollers of peristaltic pumps results in cracking and abrasion of the inner surfaces of the pump segment, leading to shedding of particles into the extracorporeal circuit.

Disclosed is a blood dialysis system including a blood dialysis machine comprising a first dialyzer, and a first phosfilter connected to the first dialyzer. In some embodiments, the phosfilter is connected to the top of the first dialyzer. In some embodiments, the phosfilter is connected to the bottom of the first dialyzer. In some embodiments, more than one phosfilter connected to the first dialyzer. In some embodiments, more than one phosfilter is connected to the blood dialysis machine. Disclosed is a blood cleansing system comprising a first dialyzer and a first phosfilter. Disclosed is a blood cleansing system comprising a first dialyzer and a first phosfilter wherein the first dialyzer is connected to the first phosfilter and a second dialyzer and a second phosfilter wherein the second dialyzer is connected to the second phosfilter. Disclosed is a blood cleansing system comprising a first dialyzer and a plurality of phosfilters.

Disclosed is a wearable artificial kidney system for a patient comprising: a volume of dialysis solution and a phosfilter. Disclosed is a wearable hemodialysis or peritoneal dialysis system for a patient comprising: a volume of dialysis solution and a phosfilter. Disclosed is a wearable peritoneal dialysis system for a patient comprising: (a) a volume of peritoneal dialysis solution that is infused into and moved out of the patient's peritoneal cavity, thereby removing from the patient waste metabolites that have diffused into the peritoneal dialysis solution; (b) a closed fluid system loop for circulating the peritoneal dialysis solution from the patient, throughout the system and back into the patient; (c) at least one pump for infusing the peritoneal dialysis solution into the patient's peritoneal cavity and moving the peritoneal dialysis solution containing waste metabolites out of the patient's peritoneal cavity and into the fluid system loop; and a phosfilter in the fluid system loop.

The dialysis treatment device comprising a phosfilter can be contained within a cartridge for a wearable artificial kidney. In an embodiment, the phosfilter is fixedly contained within the cartridge for a wearable artificial kidney. In an embodiment, the phosfilter is removably attached within the cartridge for a wearable artificial kidney.

Disclosed is a method to reduce cardiovascular morbidity and mortality in HD patients by contacting a patient's blood with a phosfilter during dialysis.

### Definitions

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity but also plural entities and also includes the general class of which a specific example may be used for illustration. The terminology herein is used to describe specific embodiments of the disclosure, but their usage does not delimit the disclosure, except as outlined in the claims.

The term “about” as used herein, in the context of any of any assay measurements refers to +/−5% of a given measurement.

As used herein, the term “adsorbent substance” refers to any substrate that adsorbs ions and/or metals from a fluid.

As used herein, the term “micro- or nano-particle” refers to polymeric particles in the micrometre size range/in the nanometer size range. A nanoparticle is typically between 1 and 100 nanometres (nm) in diameter. A microparticle is typically between 1 and 1000 micrometres (μm) in diameter.

As used herein, the term “cured polymer solution” refers to any polymer resin that is either partially reacted to become a gel or network polymer;

As used herein, the term “adsorbing complex” refers to any substance or material that physically binds or chemically bonds with a desired chemical species or complex or biological molecule.

As used herein, the term “selective adsorption” refers to an adsorptive chemical complex that has an affinity for a single chemical species, such that, only that single chemical species specifically attaches, binds or adheres to the chemical complex. For example, an adsorptive complex bound to a micro- or nano-particle surface can be designed to selectively remove at least one inorganic ion or metabolite with little to no effect on the concentrations of other physiologic blood electrolytes or other blood chemistry compounds through direct binding or adherence.

As used herein, the terms “hemoadsorption” and “hemofiltration” are indicative of removing undesired substances from blood via adsorption of the substances onto adsorptive devices and/or systems. The terms “hemoadsorption” and “hemofiltration” are synonymous terms, which may be used interchangeably in this application.

As used herein the term “selective” adsorptive compounds can include compounds having a specific chemical structure for specifically targeting, or adsorbing undesired or targeted substances, impurities or particles thereby removing the particles from an aqueous solution. For example, selective adsorptive compounds may comprise a compound having a chemical structure for specifically targeting adsorption of at least one inorganic ion or metabolite to remove at least one inorganic ion or metabolite from an aqueous solution.

As used herein, the term “adhered” as used herein, refers to any interaction between an adsorptive complex and a micro- or nano-particle. Adherence may be reversible or irreversible. Such adherence includes, but is not limited to, covalent bonding, ionic bonding, Van der Waals forces or friction, antigen-antibody interactions, and the like.

The term “biofluid” as used herein, includes any flowing material derived from an organism that contains biological materials (e.g., ions, proteins, nucleic acids etc.). For example, such biofluids may include, but are not limited to, blood, serum, dialysate, lymph, bile, saliva, urine, diarrhea, mucosal secretions, nasal secretions, cerebrospinal fluid, pus, extracellular fluid, and/or intracellular fluid.

The term “patient” or “subject”, as used herein, is a human or animal and need not be hospitalized. For example, outpatients and persons in nursing homes are “patients.” A patient may comprise any age of a human or non-human animal and therefore includes both adult and juveniles (i.e., children). It is not intended that the term “patient” connotes a need for medical treatment, therefore, a patient may voluntarily or involuntarily be part of experimentation whether clinical or in support of basic science studies.

As used herein, “Phosfilter” means a single use, disposable hemoadsorptive device that is integrated into the HD circuitry and comprises iron containing activated carbon beads in a housing with a filter at the outlet.

The term “affinity” or “selectivity” as used herein, refers to any attractive force between substances or particles that causes them to enter and remain in chemical combination. For example, a first compound that has a high affinity or selectivity for a second compound will provide greater efficacy in preventing the receptor or other compound from interacting with other compounds, than a compound with a low affinity or selectivity.

The term “derived from” as used herein, refers to the source of a compound or sequence. In one respect, a compound or sequence may be derived from an organism or particular species. In another respect, a compound or sequence may be derived from a larger complex or sequence.

### BRIEF DESCRIPTION OF THE FIGS

Preferred embodiments of the subject matter described herein will now be described with reference to the accompanying drawings, of which:

FIG. 1 shows phosphorus load and influence on adverse outcomes as well as PTH, FGF23, 1,25D, and Klotho.

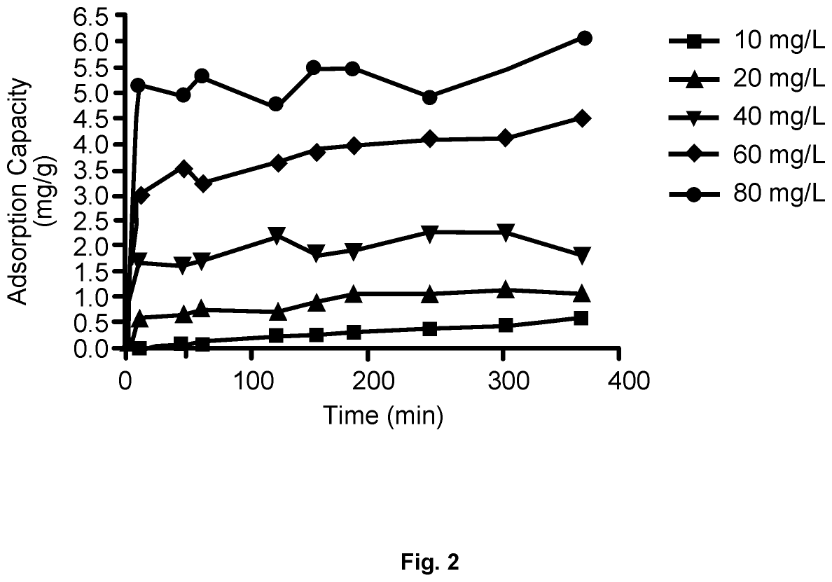

FIG. 2 demonstrates in vitro adsorption capacity of the active inorganic ion or metabolite adsorbing substrate when exposed to aqueous samples of various inorganic ion or metabolite (such as FEOOH) concentrations over time.

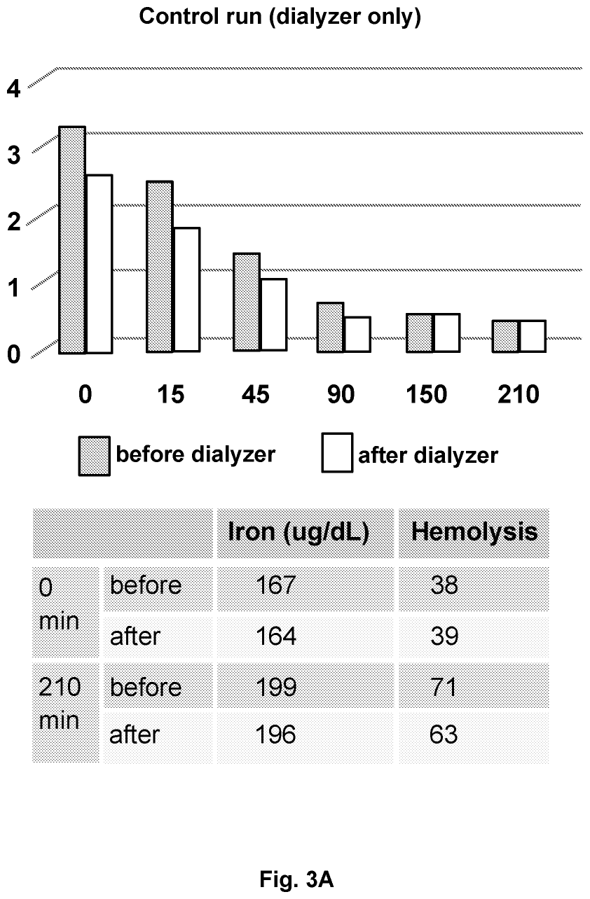

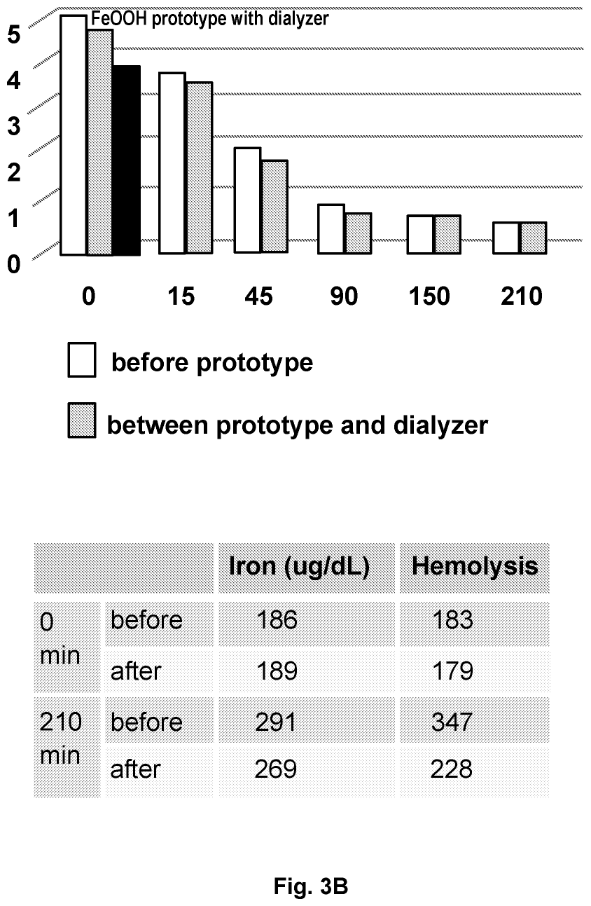

FIGS. 3A and 3B present exemplary data demonstrating the phosphate reduction by ferric oxide hydroxide adsorption complexes during in vitro blood studies with a dialyzer only and with the combination of a dialyzer with an adsorbent device. More specifically, FIG. 3 demonstrates the reduction of phosphate from blood when comparing pre and post dialyzer samples over a 4-hour timeframe, and FIG. 3B demonstrates the reduction of phosphate from blood when comparing pre and post adsorbent device samples over a 4-hour timeframe.

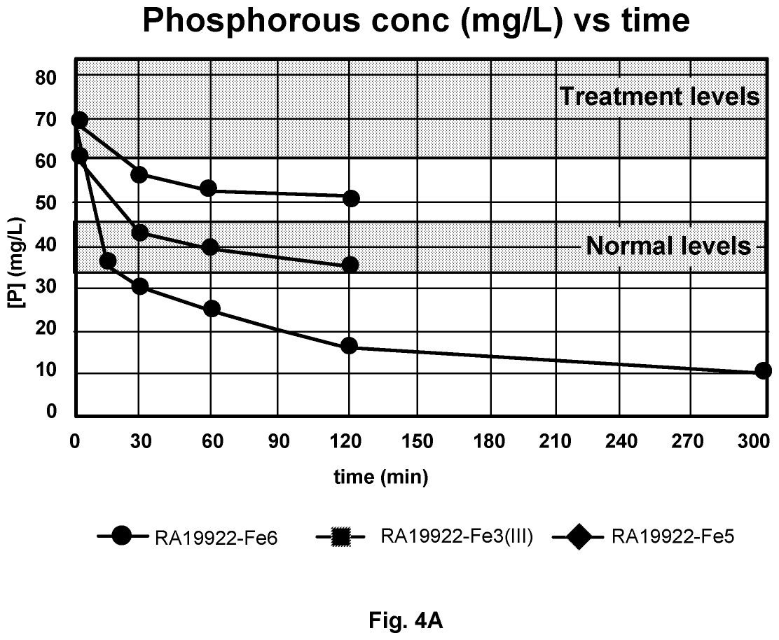

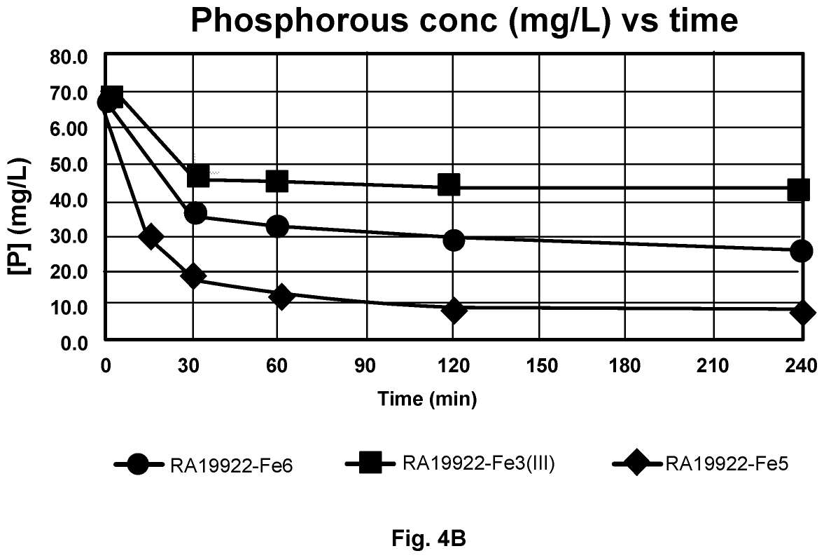

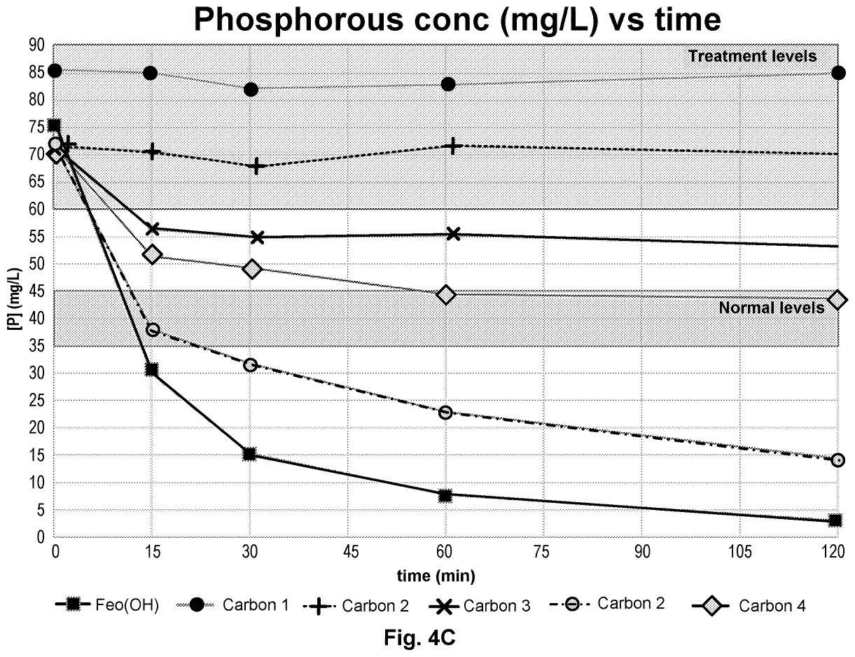

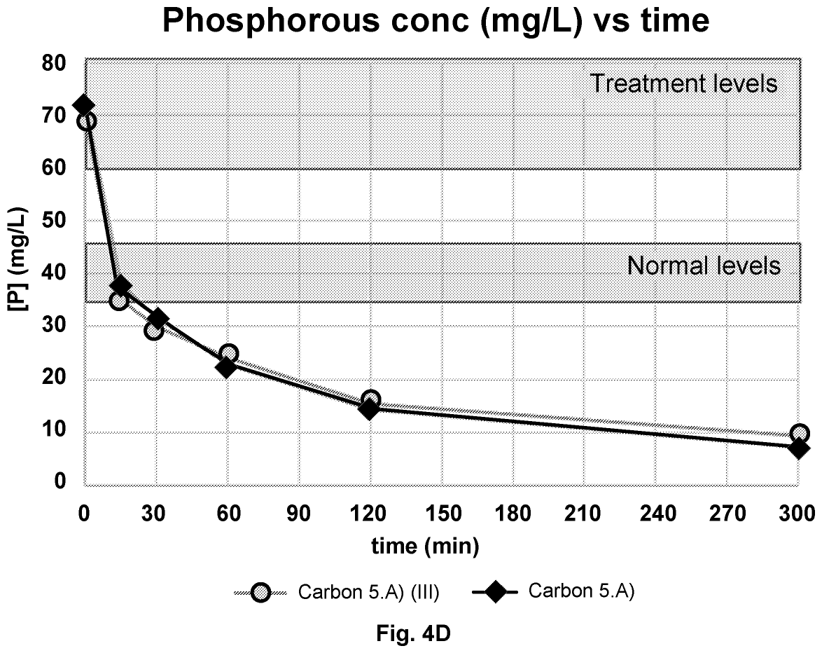

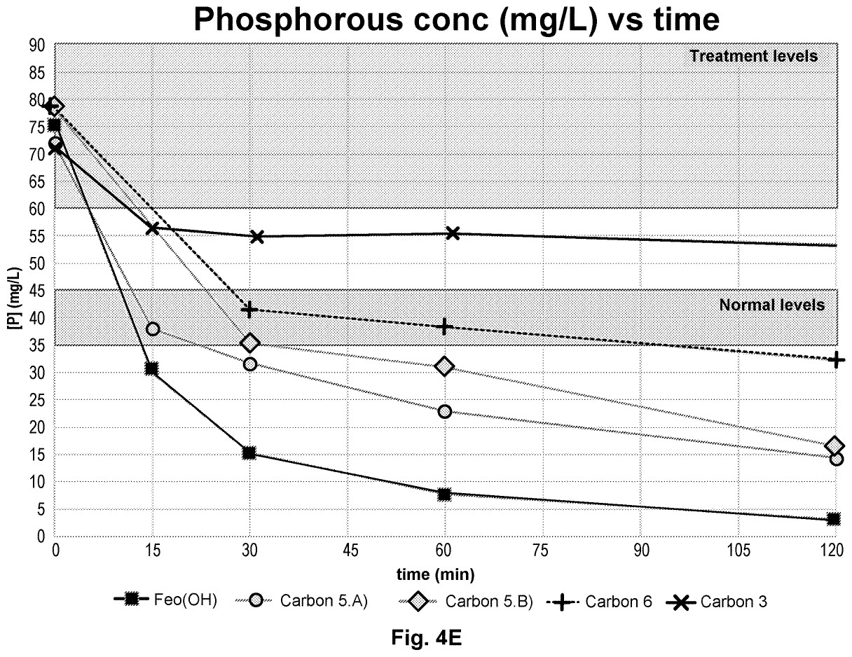

FIGS. 4A-E present exemplary data demonstrating the phosphate reduction. 4B shows phosphorous removal (II). Effect of Washing in phosphate removal. 4C shows phosphorous removal (I). 4D shows phosphorous removal (II). effect of washing in phosphate removal. 4E shows phosphate removal (III). reproducibility and effect of iron salt concentration.

FIG. 5 presents exemplary data showing phosphate removal over time during in vivo studies of intermittent hemodialysis in healthy canines. Sampling of phosphate concentrations from blood occurs at dialyzer inlet and outlet.

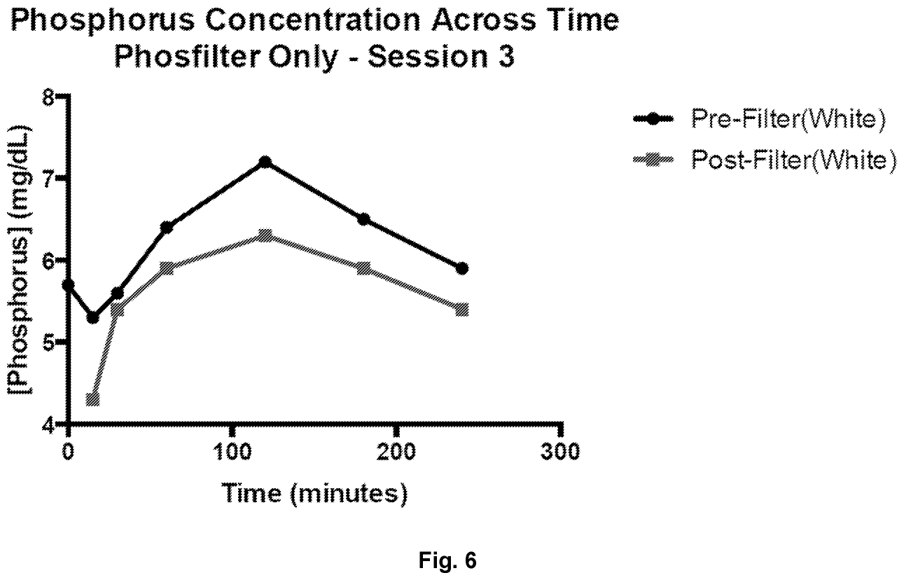

FIG. 6 presents exemplary data from healthy canines following use of an adsorptive device (phosfilter) by itself in a hemodialysis circuit.

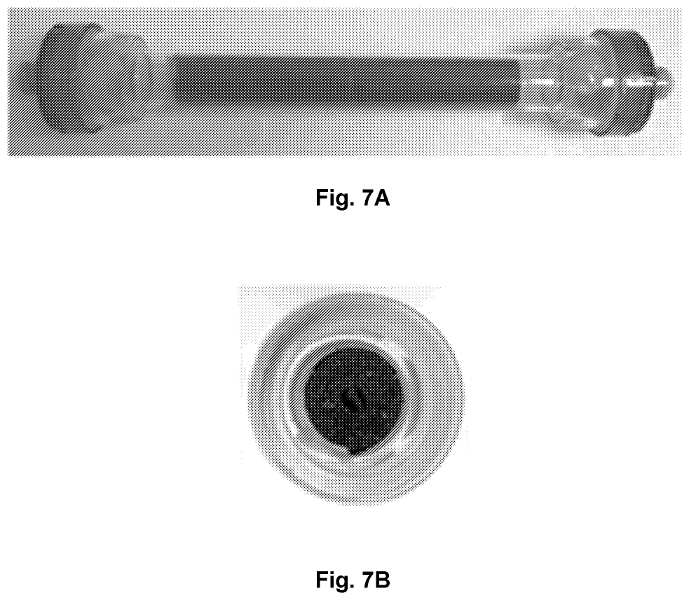

FIGS. 7A-B represent the adsorbent device prototype. Views represent a side view of the device (7A) and a view of the coiled interior (7B) that contains the “active” moiety that selectively adsorbs phosphates.

FIG. 8 represents the conventional hemodialysis circuitry. The hemoadsorption device is placed upstream of the dialyzer. The hemoadsorption device could also be placed downstream of the dialyzer. 8.1 represents the hemadsorption device. 8.2 represents a port for pressure monitoring. 8.3 represents the top/upper lid to the hemoadsorption device. 8.4 represents the inlet for blood. Blood tubing with a luer lock would connect to the inlet and outlet. 8.5 represents the body of the hemoadsorption device. In some embodiments, the device comprises FeOH containing beads inside of the device. 8.6 represents the bottom/lower lid to the hemoadsorption device. The lower lid (just above the outlet for blood) comprises a filter to prevent release of particles >10 microns 8.6 represents the outlet of blood. In some embodiments, the phosfilter is connected to the dialyzer at port 8.6. In some embodiments, the phosfilter is connected to the dialyzer at port 8.4.

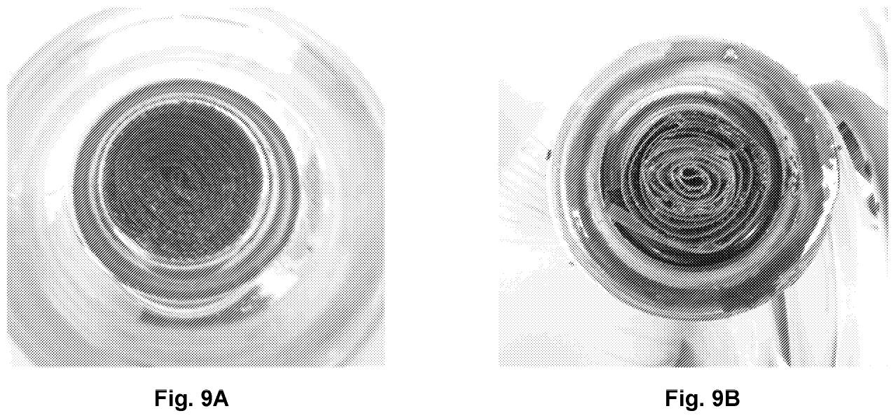

FIGS. 9A and 9B show embodiments of the adsorbent device with mesh (8A) and silicone (8B).

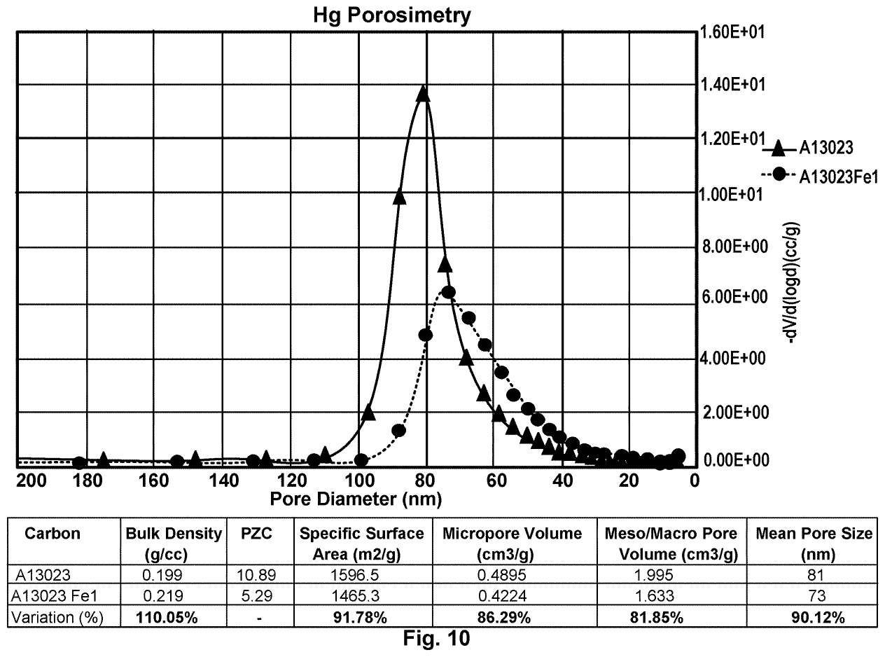

FIG. 10 present exemplary data demonstrating pore structure of support carbon (A13023) and carbon containing bound iron (A13023-Fe1). The graph represents the pore size distribution of meso- and macro-pores for both samples. The table shows bulk density and surface properties of samples and differences due to iron loading: PZC=point of zero charge.

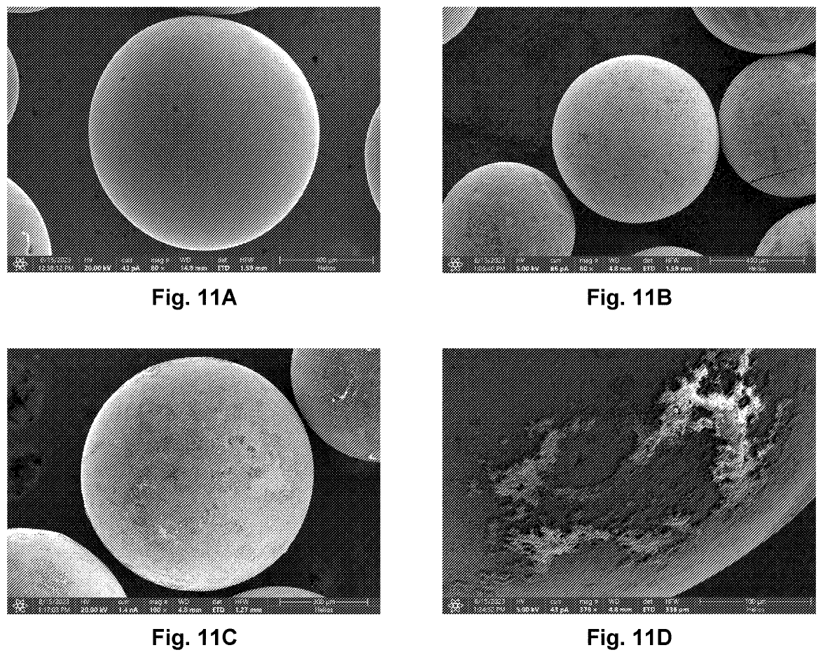

FIGS. 11A-D. presents exemplary data showing scanning electron microscopy of carbon beads. A) Activated carbon beads without Iron functionality to remove Pi. B) Activated carbon beads with iron functionality designed to remove Pi. C) Activated carbon beads with iron functionality after exposure to bovine blood for 4 h at 37 C. D) Magnification of C showing blood cells.

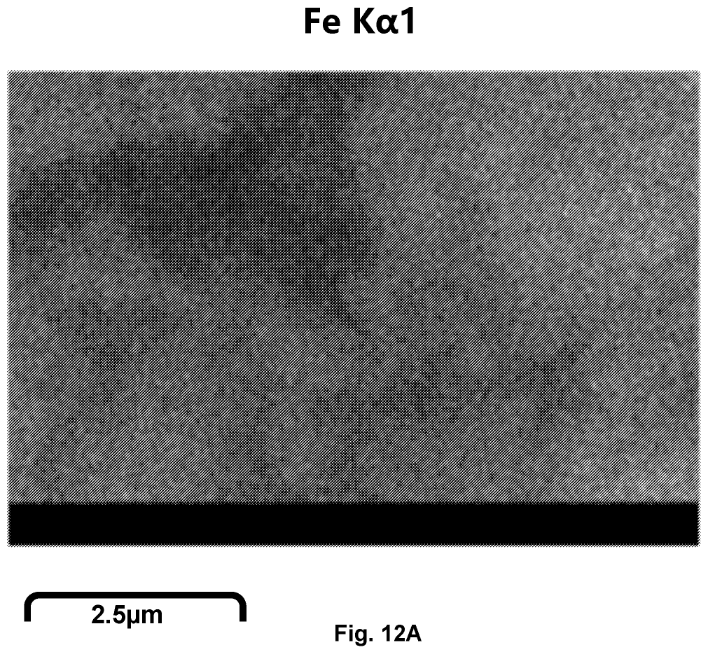

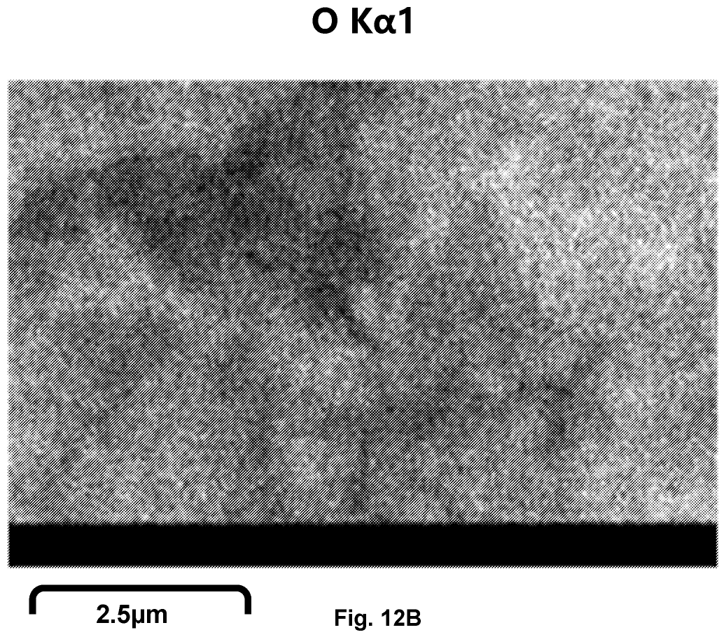

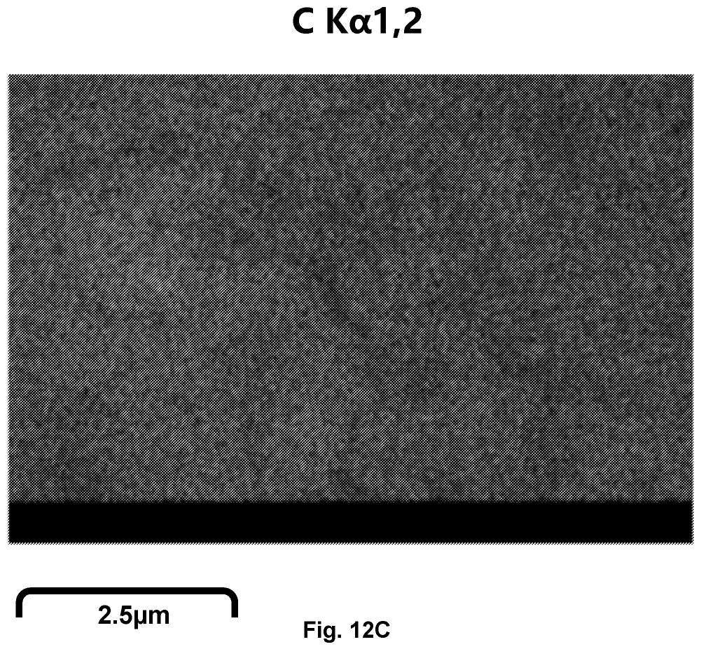

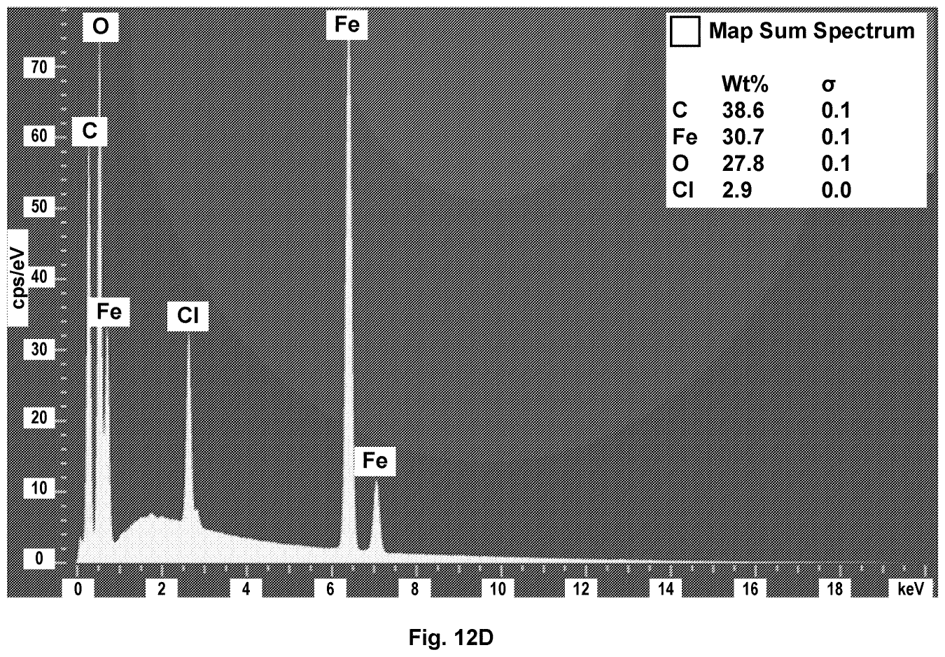

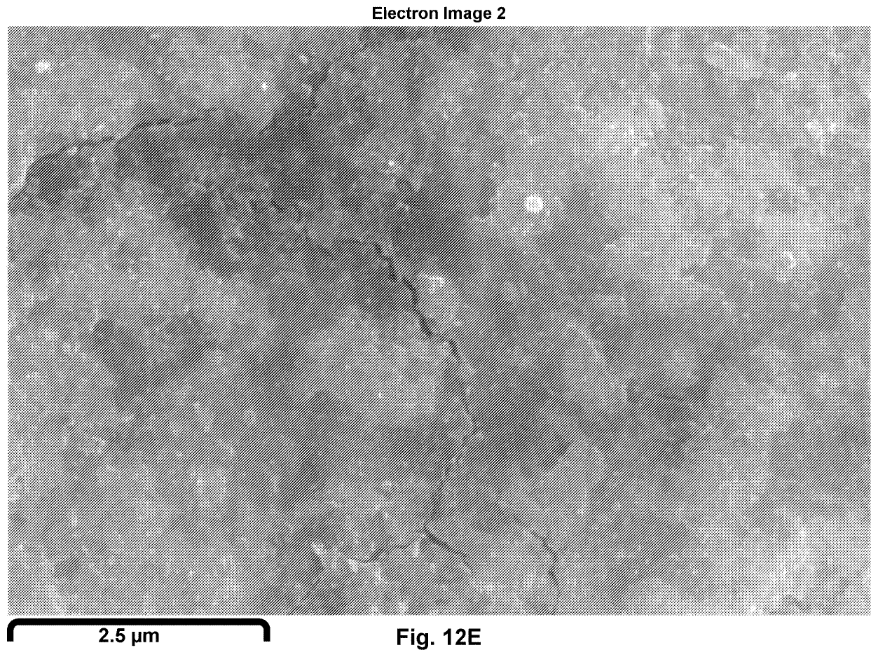

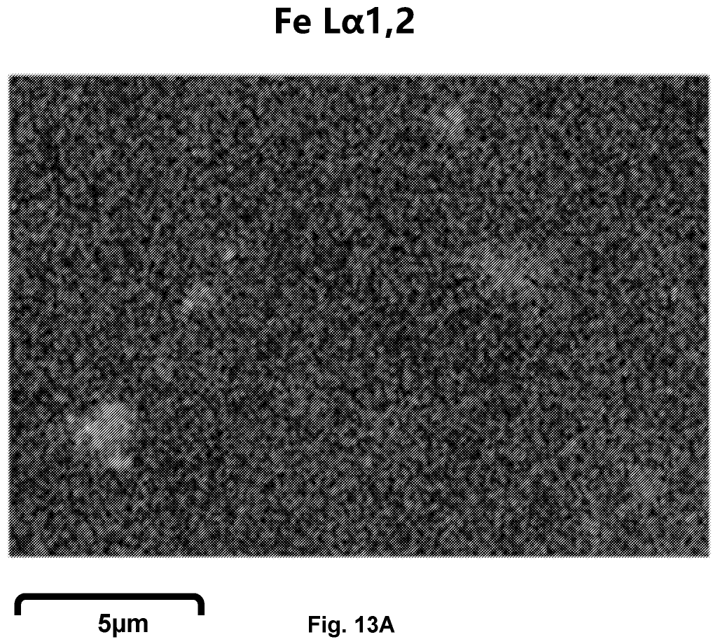

FIGS. 12A-E show an embodiment of activated carbon A13023-Fe1 prior to blood exposure and washed. FIGS. 12A-C show activated carbon A13023-Fe1 with iron, oxygen and carbon presence primarily on the bead surface. EDS graphic (FIG. 12D) shows the weight % of each element. FIG. 12E shows the surface of the activated carbon. These results show modifications to the activated carbon that incorporate iron predominantly on the exterior to adsorb blood phosphate.

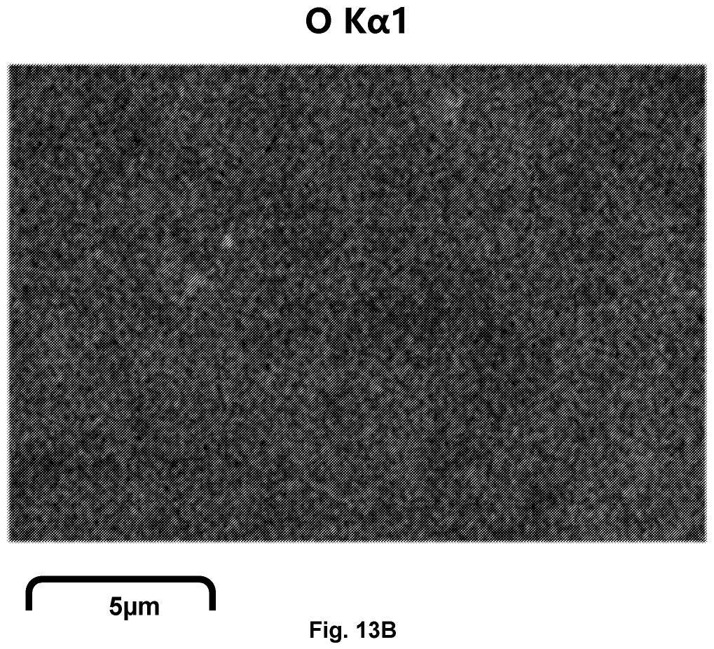

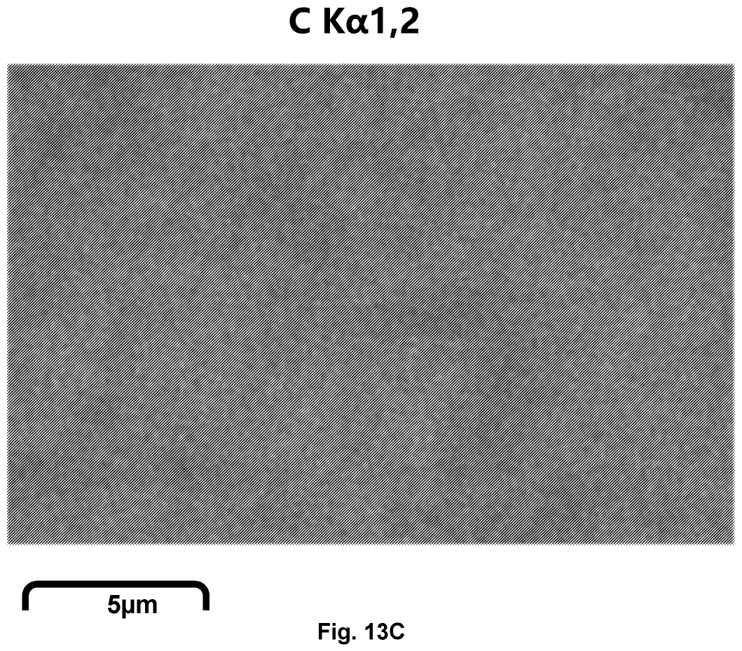

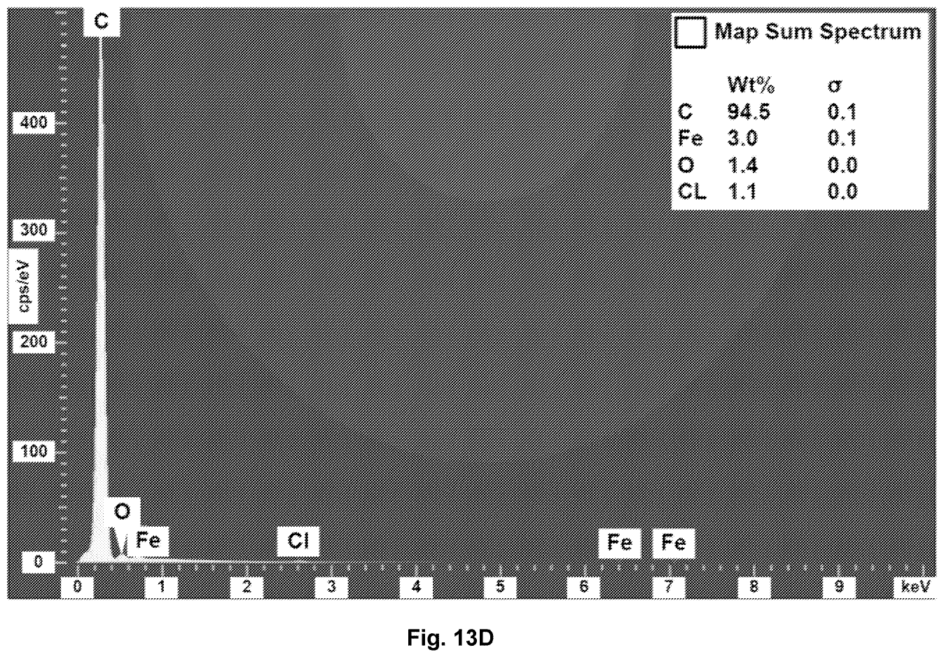

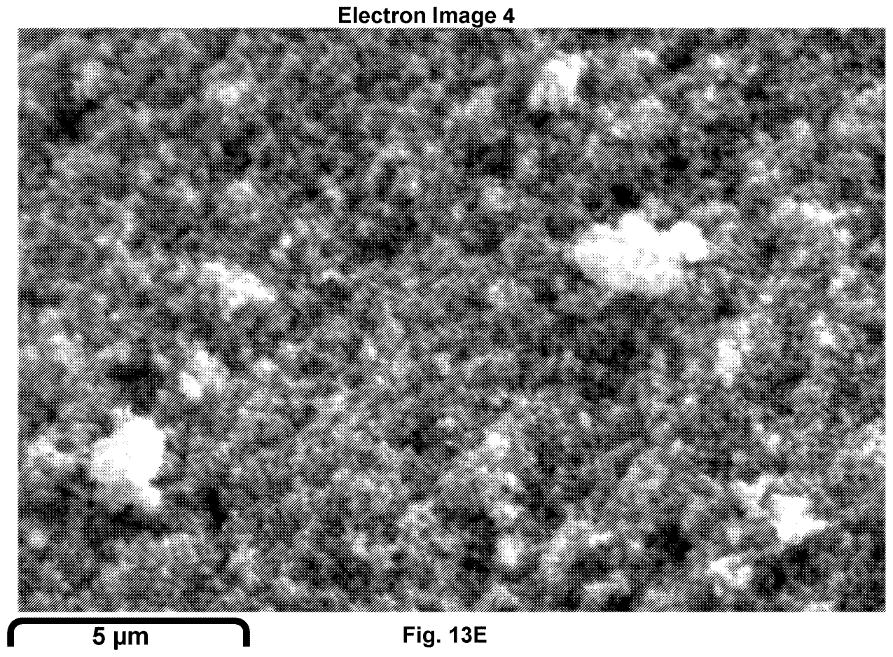

FIGS. 13A-E show an embodiment of activated carbon A13023-Fe1 prior to blood exposure and washed. FIGS. 13A-C show activated carbon A13023-Fe1 with iron, oxygen and carbon presence in the bead interior. EDS graphic (FIG. 13D) shows the weight % of each element. FIG. 12E shows the interior of the activated carbon. These results suggest the potential for more iron loading on the interior.

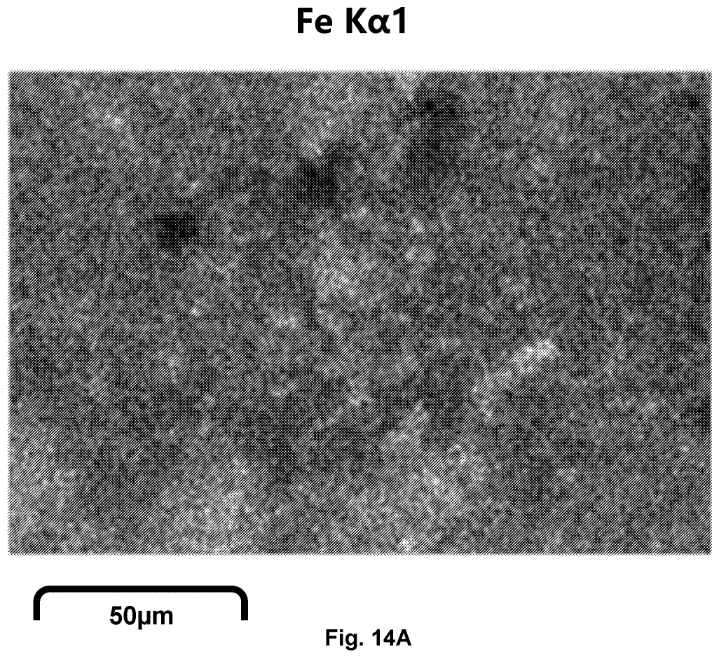

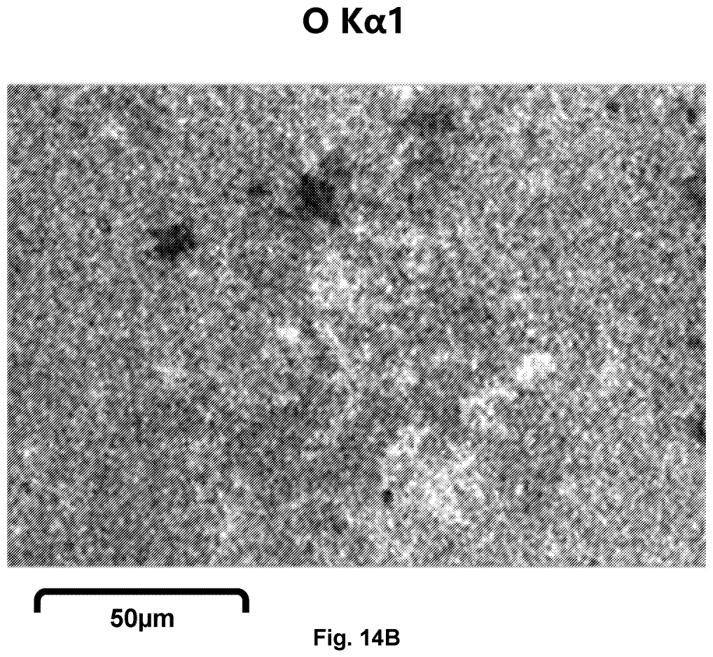

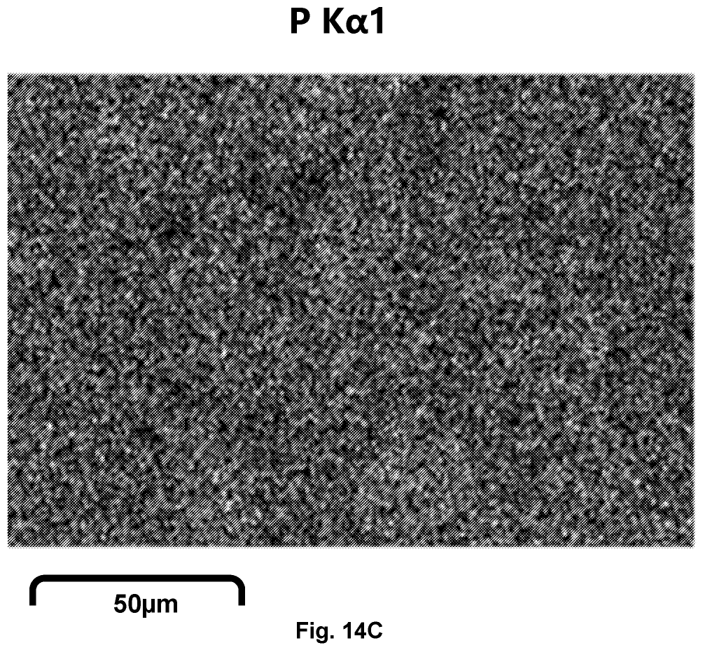

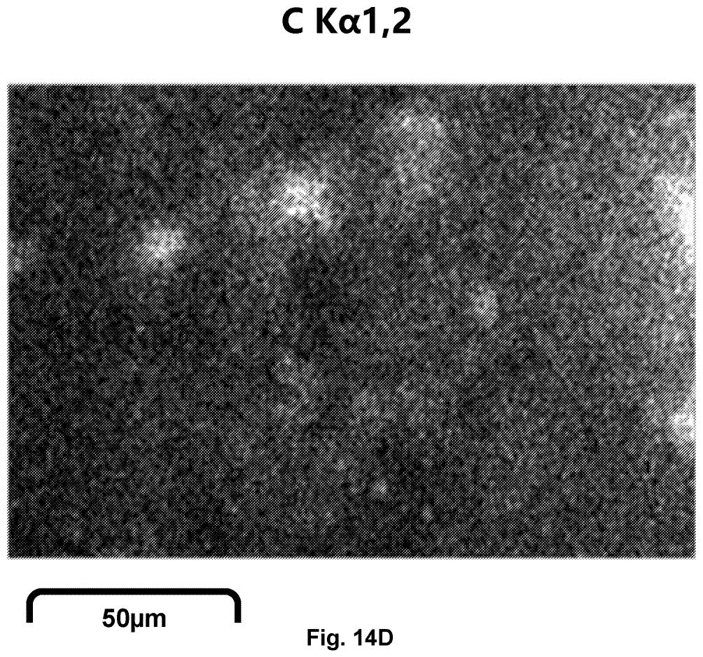

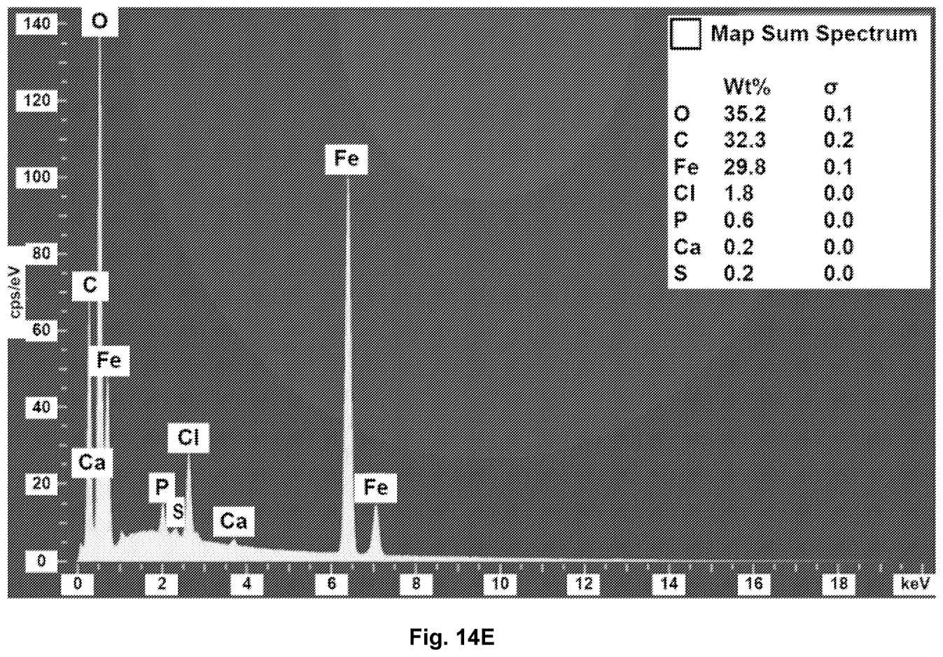

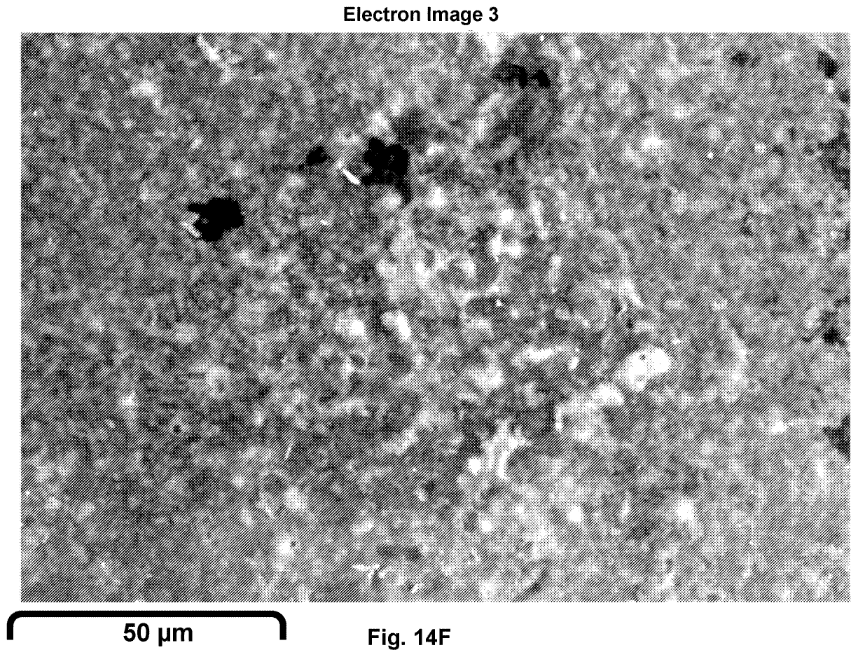

FIGS. 14A-F show an embodiment of activated carbon A13023-Fe1 used in whole blood and washed. FIGS. 14A-C show activated carbon A13023-Fe1 after exposure to bovine blood for 4 hours at 37 degrees C. with iron, oxygen, carbon and phosphate presence primarily on the bead surface. Additionally, microscopy supports the presence of phosphate primarily on the bead surface in a similar pattern to iron (FIG. 14D). EDS graphic (FIG. 14E) shows the weight % of each element. FIG. 14F shows the surface of the activated carbon. These results suggest that modifications that incorporate iron on the exterior will further enhance phosphate adsorption.

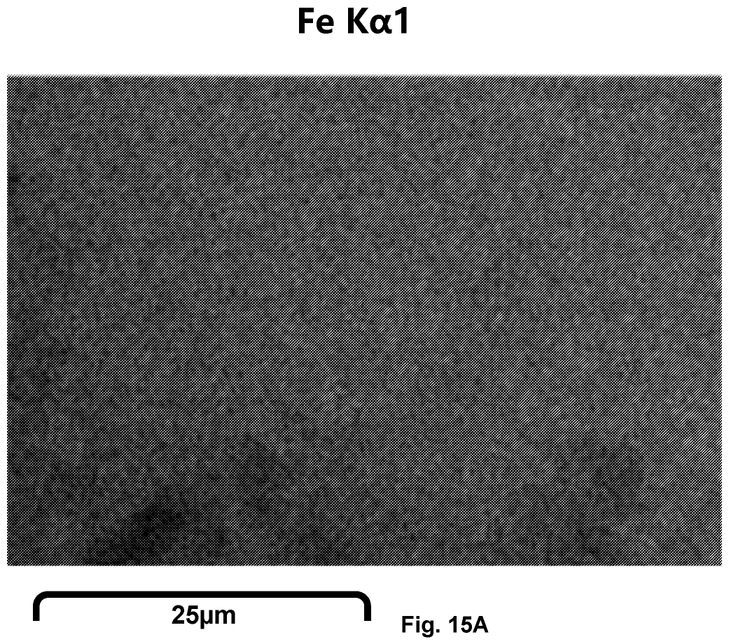

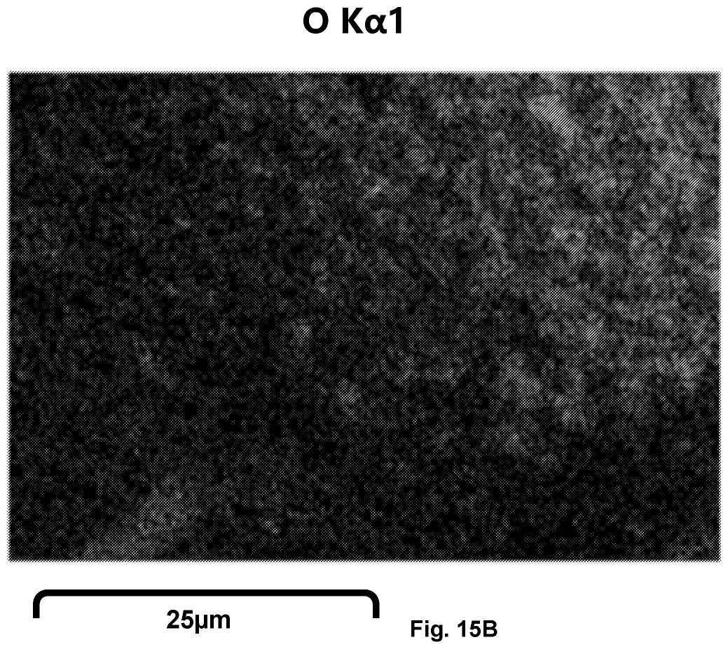

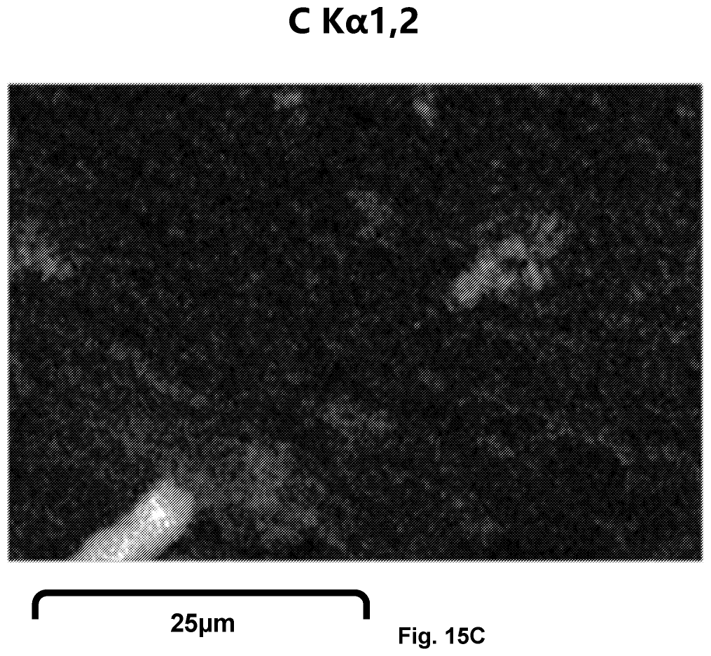

FIGS. 15A-E show an embodiment of activated carbon A13023-Fe1 used in whole blood. FIGS. 15A-D show activated carbon A13023-Fe1 after exposure to bovine blood for 4 h at 37 degrees C. with iron, oxygen, carbon and phosphate presence in the bead interior. FIG. 15E shows the interior of the activated carbon. These results suggest that modifications that incorporate iron on the exterior will further enhance phosphate adsorption.

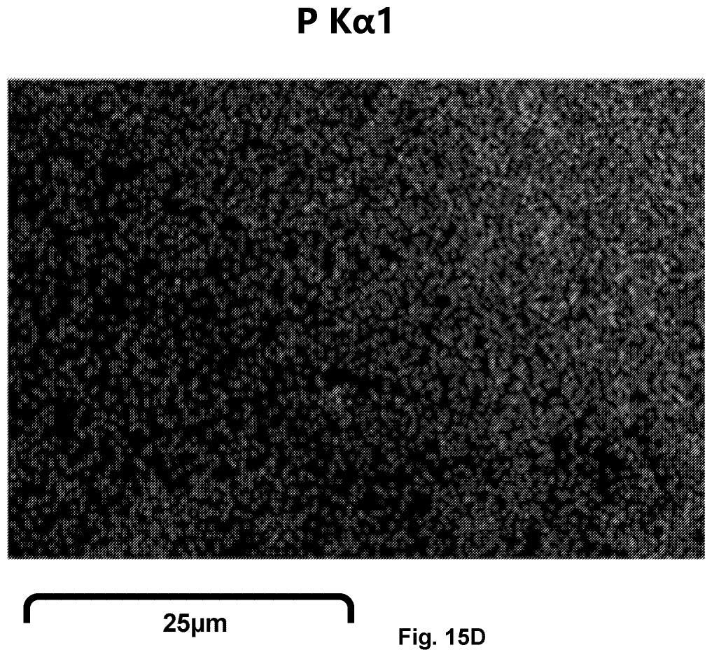

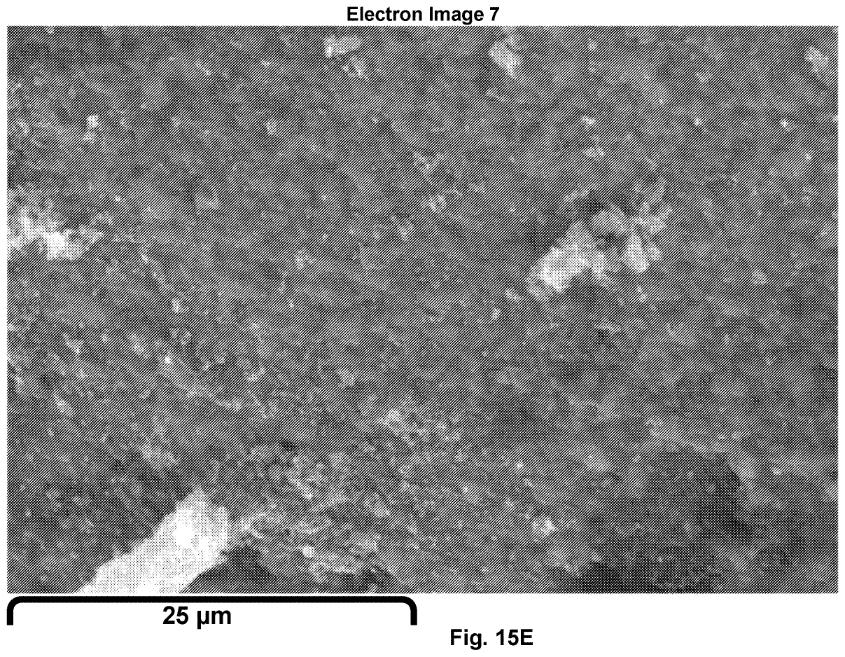

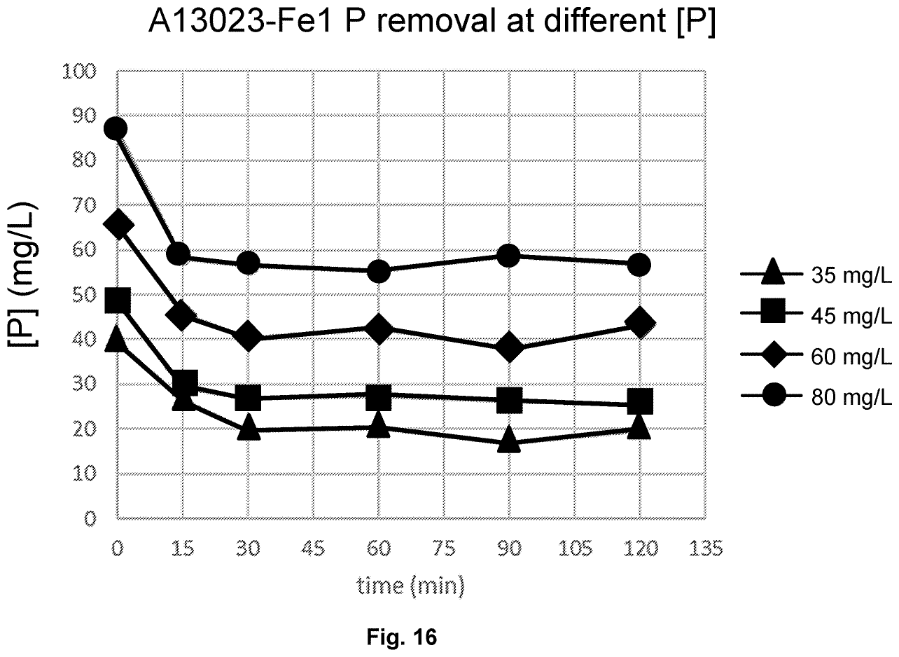

FIG. 16 shows exemplary data of removal of phosphate by the Phosfilter. Solutions containing phosphate at 35, 45, 60 and 80 mg/L were exposed to A13023-Fe beads in a beaker at room temperature and evaluated over 2 hours.

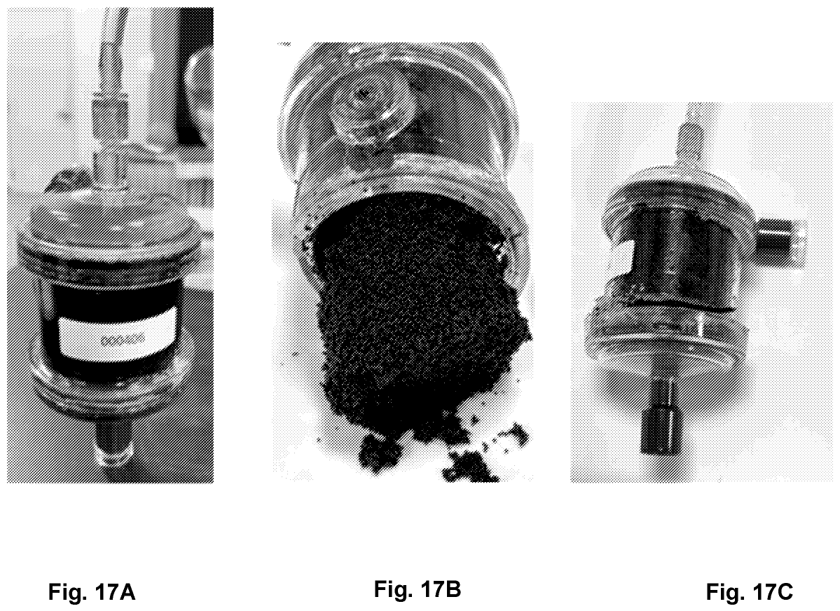

FIGS. 17A-C shows an exemplary phosfilter (closed-17A), open (17B), perspective view (17C). The phosfilter resembles a hemofilter. The Phosfilter shown comprises phosphate adsorbing carbon particles. As seen in FIGS. 17A-C the phosfilter is filled with beads (iron containing activated carbon beads) in the interior of the device which is the “active” moiety that selectively adsorbs phosphates.

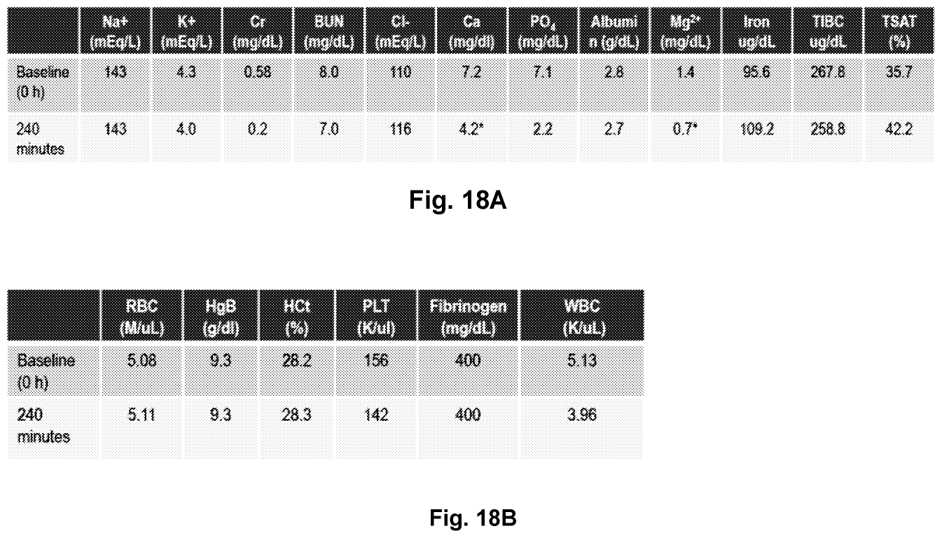

FIGS. 18A-B present exemplary data of a chemistry panel for bovine blood at baseline and 4 hours (18A) and CBC Panel for Bovine Blood at Baseline and 4 Hours (18B). A mitigation strategy was subsequently employed whereby the Phosfilter was stored prior to use in a solution of calcium to bind non-specific pores on the carbon particles. A subsequent 4 h benchtop study conducted in a phosphate solution showed mitigation of reductions in calcium and magnesium, while retaining reductions in phosphate.

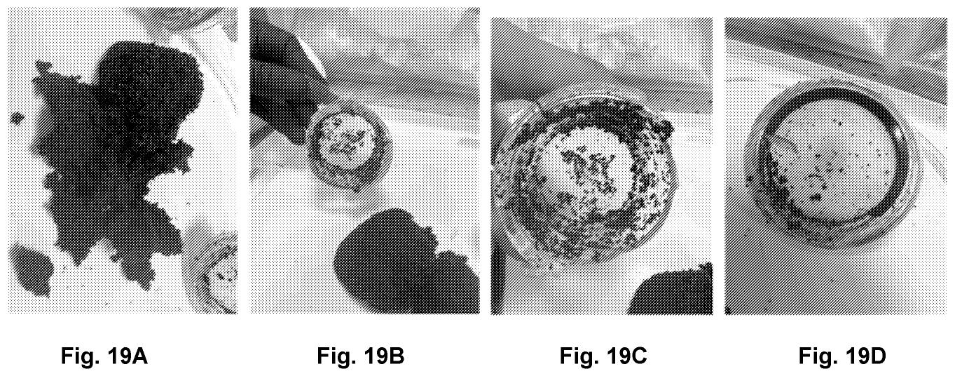

FIGS. 19A-D shows pictures representing the interior of a phosfilter device after exposure to blood and rinsed. The activated carbon beads show integrity and without the presence of interspersed clots (FIG. 19A). FIGS. 19B and C represent the top end of the device, demonstrating limited clotting and contact with the housing. FIG. 19D shows the bottom of the device with a filter and lack of presence of blood clots.

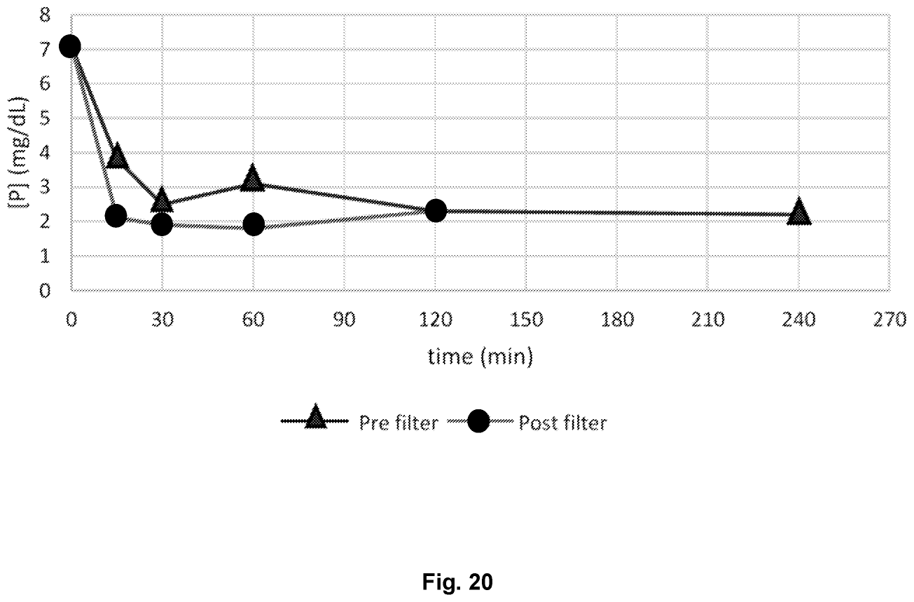

FIG. 20 present exemplary data showing phosphate concentrations in bovine blood. It demonstrates phosphate reduction by ferric oxide hydroxide adsorption complexes during ex vivo blood studies with a hemoadsorption device alone (phosfilter). More specifically, FIG. 20 demonstrates the reduction of phosphate from blood when comparing pre and post phosfilter samples over a 4-hour timeframe. Fresh bovine blood (1.5 L) was pumped through a circuit containing the Phosfilter for 4 h at 37 C. Samples (6 mL) were obtained pre- and post-device, centrifuged and serum assayed for phosphate concentrations. Average percent reduction was 44% over 60 min. The phosphate nadir was maintained throughout the remaining 3 h of the experiment. The data demonstrated a 4.9 mg/dL reduction in serum phosphate concentration during the 4 h ex vivo study. Experiment was run with just a blood pump and not in combination with the dialyzer.

FIG. 21 shows exemplary data of indoxyl sulfate reduction with the phosfilter.

FIG. 22 shows exemplary data that demonstrates the relationship between increases in serum phosphate and mortality. Specifically, it shows the stepwise increase in relative risk of death at serum phosphate concentrations above 5 mg/dl. The data is adjustment for age, gender, race or ethnicity, diabetes, and vintage; Block, GA JASN; 15:2208-18.

### DETAILED DESCRIPTION

The present disclosure is related to chemical adsorption devices, systems, and methods. In particular, a chemical adsorption device includes, but is not limited to, a micro- or nano-particle and an adsorbing complex, the adsorbing complex being reactive to selectively adsorb a substance from a fluid passing over and/or through the micro- or nano-particle, to remove at least a portion of the substance from the fluid. The micro- or nano-particle of the device further includes a micro- or nano-particle and an activated carbon, the micro- or nano-particle and activated carbon being contained in a cylindrical housing. See FIGS. 8 at 8.1-8.7, and 17A-C.

In accordance with the subject matter disclosed herein, adsorption devices, systems, and methods are provided. Reference will now be made in detail to possible embodiments or embodiments of the subject matter herein, one or more examples of which are shown in the figures. Each example is provided to explain the subject matter and not as a limitation. In fact, features illustrated or described as part of one embodiment can be used in another embodiment to yield still a further embodiment. It is intended that the subject matter disclosed and envisioned herein covers such modifications and variations.

### I. Hyperphosphatemia

Hyperphosphatemia occurs in 90% of hemodialysis patients. However, despite current treatments comprised of oral phosphate binders, 50% of patients fail to reduce blood phosphate concentrations to clinically acceptable levels (e.g. 3.5 to 5.5 mg/dL) and represent the difficult to control patients. It is established that phosphates are considered to be uremic toxins, along with other metabolites such as indoxyl sulfate. Recent studies have illustrated clear associations between mortality and hyperphosphatemia. It is evident that hyperphosphatemia significantly contributes to the 10-20 fold higher rate of cardiovascular death in ESRD as compared to age-matched controls. Although oral phosphate binding drugs exist, there are many problems associated with drugs, for example:
1) Fundamental 40% capacity limitation to bind phosphorus in the intestine;
2) Drug side effect of elevation in blood calcium leading to worsening calcification through complexation with phosphates in the blood vessels;
3) Other drug side effects of diarrhea, constipation, and off-target binding of vitamins in the diet that can lead to adverse effects, such as pernicious anemia;
4) Poor patient medication compliance as a result of complicated medication regimens, lack of understanding of the need for therapy, lack of convenience, high out-of-pocket costs, and side effects;
5) Lifestyle issues such as dietary restrictions and associated burden of the need to take as many as 16 pills per day in addition to other prescribed medications;
6) Potential aluminum toxicity by the aluminum-based oral phosphate binding drugs.

The present disclosure contemplates various interventions that are urgently needed to control hyperphosphatemia given the limited efficacy and adverse consequences of current approaches.

### Uremic Toxins

Uremic toxins are a heterogeneous group of metabolite molecules that accumulate in the body due to the progression of chronic kidney disease. These metabolites are associated with kidney dysfunction and the development of comorbidities in patients with CKD, with only some being only partially eliminated by dialysis therapies. One of the main consequences of the loss of renal function is an accumulation of uremic toxin metabolites in the body, affecting the various tissues and organs, including the cardiovascular system. The biological effects promoted by uremic toxin metabolites depend on the relationship between production, degradation, and excretion, in addition to cytoplasmic distribution and the presence of inhibiting or promoting agents of the toxin's action. The European Uremic Toxin Work Group (EUTox) reports that uremic toxin metabolites can be classified into three groups due to their physicochemical characteristics and their behavior during dialysis: (I) small-water soluble compounds (molecular weight 500 Da), such as creatinine and urea which are easily removed by hemodialysis; (II) medium compounds (peptides with molecular weight >500 Da), such as cystatin-C and β2-microglobulin, which can only be removed by large pore size dialysis membranes during hemodialysis; and (III) protein-bound uremic toxins (PBUTs), such as indoles and phenols, which come from dietary amino acid metabolism and are poorly filtered by the dialytic membrane in hemodialysis. These latter uremic toxin metabolites include such compounds as indoxyl sulfate. Patients with CKD have a total indoxyl sulfate concentration surpassing 500 μM compared to 0.1-2.39 μM in patients with healthy kidney functions. The lack of removal by dialysis in CKD is associated with diverse harmful effects in other organs, such as alterations to thyroid function, endothelial dysfunction, smooth muscle cell proliferation, and atherosclerosis. Indoxyl sulfate is related to many harmful effects to the organism, with a hypertrophic effect in cardiomyocytes through the activation of the mitogen-activated protein kinase (MAPK) and nuclear factorκB (NF-κB) pathways among them, in turn indicating that this toxin has a crucial role in developing cardiac hypertrophy under uremic conditions. Another effect of indoxyl sulfate is the activation of proinflammatory macrophages which generate an immune dysfunction. There are many more uremic toxin metabolites, that similar to indoxyl sulfate, are not effectively removed by dialytic methods in patients with CKD.

### II. Inorganic Ion or Metabolite Adsorbent Devices

According to one embodiment, the subject matter described herein includes an inorganic ion or metabolite adsorption device. The inorganic ion or metabolite adsorption device can comprise an adsorbent substance and an adsorbing complex adhered to the adsorbent substance. In one embodiment, the adsorbent substance is a micro- or nano-particle, and the adsorbing complex is adhered to a surface of the micro- or nano-particle. In one embodiment, the adsorbent substance is a bead material. In one embodiment, the adsorbent substance is a micro- or nano-particle. In one embodiment, the adsorbent substance is a resin. In one embodiment, the adsorbent substance is housed in a cartridge. Although it is not necessary to understand the mechanism of an invention, it is believed that the adsorbing complex may be adapted to selectively adsorb specific undesirable inorganic ions present in a biofluid. In one embodiment, the inorganic ion may include anions and cations. In one embodiment, undesired concentrations of other substances (e.g., for example, non-selected ionic chemical species) found in bodily fluids, for example, blood can be filtered via adsorptive devices, methods, and systems described. See FIG. 6 showing data from healthy canines following use of an adsorptive device (phosfilter) by itself in a hemodialysis circuit (Pre and post device phosphate data from one canine only).

Referring now to FIGS. 7 and 8 an adsorption device is illustrated. In one embodiment, the present disclosure contemplates an adsorption device comprising an adsorbent micro- or nano-particle. In one embodiment, the present disclosure contemplates a method of creating an adsorption device comprising; i) contacting a micro- or nano-particle with a cured polymer resin and 2) adhering an adsorbing complex to create an adsorbent surface. In one embodiment and without limitation, an adsorbent surface can comprise a micro- or nano-particle adhered to ferric oxide hydroxide (FeOOH) and may include an activated carbon.

In one embodiment, an adsorptive complex can include, but is not limited to, at least one compound for selectively adsorbing phosphate ions or metabolites. In one embodiment, at least one compound comprises ferric oxide hydroxide (FeOOH) and an activated charcoal. In one embodiment, the adsorption device can comprise an adsorbent hemofiltration micro- or nano-particle. Although it is not necessary to understand the mechanism of an invention, it is believed that the adsorbent hemofiltration micro- or nano-particle can be used for filtering blood during hemodialysis such that phosphate ions or metabolites are adsorbed onto the micro- or nano-particle and effectively removed from the blood.

In one embodiment, the present disclosure contemplates a hemoadsorption system comprising an adsorption device having at least one adsorbent complex. In one embodiment, the hemoadsorption system effectively treats hyperphosphatemia in patients (e.g., FIG. 8)

In one embodiment, the hemoadsorbent system effectively treats sepsis or septic shock. In one embodiment, the hemoadsorbent system effectively treats acute liver failure. Other embodiments include the removal of metabolites, cells, toxins, drugs, antigens and antibodies. Such hemoadsorbent systems may comprise adsorption devices containing micro- or nano-particles that are capable of adsorbing harmful particles and/or contaminants onto the micro- or nano-particle to effectively remove these contaminants from the body and/or bodily fluids. Adsorption devices, systems, and methods described herein can be used to treat any contaminated or potentially contaminated biofluid.

### A. Micro- or Nano-Particles

Micro- or nano-particle can comprise any type of biocompatible polymer. In one embodiment, existing micro- or nano-particle can, for example and without limitation, comprise a medical grade silicone. However, any suitable biocompatible polymer is contemplated including, but not limited to, polyethylene glycol, co-glycolides, co-lactides, polyesters, polyamides, vinyl polymers, and manufactured cellulosics. In some embodiments, the particles are 500-1000 microns or 500,000 to 1,000,000 nm.

When a micro- or nano-particle undergoes adsorbent surface, interior, or both treatment, it can transform into an adsorbent micro- or nano-particle, or adsorbent device enhanced, for example, by a permanently bound phosphate or metabolite selective adsorptive mechanism or complex. Adsorbent treatment (surface, interior, or both) can comprise a surface treatment, interior treatment or both to secure adsorptive compounds to micro- or nano-particle, thereby forming an inorganic ion-selective or metabolite adsorption device. Substances adsorbed by an adsorbing complex can, for example, comprise inorganic ions at unwanted (e.g., toxic) concentrations including, but not limited to, phosphate, chloride, sodium, ferric, calcium, magnesium and/or zinc. In one embodiment, an adsorbent surface, interior, or both treatment can transform existing micro- or nano-particle into an adsorbing device comprising a medical grade silicone coated with a cured polymer solution and phosphorus-selective adsorbent complex. Although it is not necessary to understand the mechanism of an invention, it is believed that an adsorption device may be used to specifically remove phosphate ions, or particles from a biofluid or dialysate.

### B. Micro- or Nano-Particles

Micro- or nano-particles can comprise any type of biocompatible substance. However, any suitable biocompatible micro- or nano-particle is contemplated including, but not limited to, an activated charcoal, anion exchanger, or peptides.

When a micro- or nano-particle undergoes adsorbent surface, interior, or both treatment, it can transform into an adsorbent micro- or nano-particle, or adsorbent device enhanced, for example, by a permanently bound phosphate or metabolite selective adsorptive mechanism or complex. Adsorbent surface, interior, or both treatment can comprise a surface treatment to secure adsorptive compounds to micro- or nano-particles, thereby forming an inorganic ion-selective or metabolite adsorption device. In some embodiments, substances adsorbed by an adsorbing complex can, for example, comprise inorganic ions at unwanted (e.g., toxic) concentrations including, but not limited to, phosphate, creatinine, and other uremic toxins. HD patients do not want reduced calcium as they tend to have low calcium. In some embodiments, substances adsorbed by an adsorbing complex can, for example, comprise inorganic ions at unwanted (e.g., toxic) concentrations including, but not limited to, phosphate, chloride, sodium, ferric, calcium, magnesium, zinc, other metals or metabolites. Patients with tumor lysis syndrome want calcium reduction. In one embodiment, an adsorbent surface, interior, or both treatment, interior treatment or both, can transform existing micro- or nano-particles into an adsorbing material comprising a phosphorus-selective or metabolite adsorbent complex. Although it is not necessary to understand the mechanism of an invention, it is believed that an adsorption device may be used to specifically remove phosphate ions or metabolites, or particles from a biofluid or dialysate.

### C. Adsorbent Complex Preparation

In one embodiment, the present disclosure contemplates an ionic adsorbent complex. In one embodiment, the ionic adsorbent complex is ferric oxide hydroxide (FeOOH). See, Table 1.

TABLE 1
The chemical structure for FeOOH.

| Chemical Name: | Ferric Oxide Hydroxide |
|---|---|
| CAS Registration Number: | 1310-14-1 |

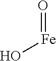

Although it is not necessary to understand the mechanism of an invention it is believed that ferric oxide hydroxide (FeOOH) effectively and selectively adsorbs phosphate from a surrounding environment (e.g., for example, a bodily fluid), thereby reducing the concentration of phosphate ion in the surrounding environment.

In one embodiment, ferric oxide hydroxide (FeOOH) can be adhered to a micro- or nano-particle during adsorbent surface, interior, or both treatment to create an adsorption device, or adsorbent micro- or nano-particle, wherein a ferric oxide hydroxide (FeOOH) can be adhered to commercially available s micro- or nano-particles using any suitable process. In one embodiment, processes using heat and pressure may be used to adhere ferric oxide hydroxide (FeOOH) onto micro- or nano-particles.

In one embodiment, a method of preparing the adsorbent complex comprises adhering an adsorption complex to a micro- or nano-particle to create an adsorbent micro- or nano-particle. In one embodiment, the step of adhering a ferric oxide hydroxide (FeOOH) adsorbent complex to a micro- or nano-particle can be prepared through chemistry. The solution can be allowed to react and loaded or coated with ferric oxide hydroxide (FeOOH).

Prepared adsorption micro- or nano-particles can be considered sufficiently coated when ferric oxide hydroxide (FeOOH) substantially covers the entire surface, interior, or both of the micro- or nano-particles. Prepared adsorption micro- or nano-particles can be further rinsed and sterilized with deionized water until no loose particles or reddish color are present.

The prepared adsorbent micro- or nano-particle can then be dried and a final mass can be measured and recorded. The micro- or nano-particles secured with ferric oxide hydroxide (FeOOH) can be used alone and/or in combination with other devices to adsorb impurities, such as phosphates, from any suitable biofluid including, but not limited to blood. Optionally, adsorbent micro- or nano-particle can be positioned and/or placed in a readily available housing unit, for example, like a dialyzer housing for dialyzing blood.

In one embodiment, and with reference to FIG. 9A, the device may comprise mesh. In one embodiment, and with reference to FIG. 9B, the device may comprise silicone.

In one embodiment, a method of preparing the adsorbent complex comprises adhering the adsorption complex to a micro- or nano-particle using similar steps as described above. These particles may contain an activated charcoal.

In some embodiments, disclosed is a method for extracorporeal treatment of blood to remove a contrary substance therein. In some embodiments, the method further comprises providing the treated blood back to the body (return). In some embodiments, the method further comprises, contacting the blood with a material comprising a coated microporous/mesoporous or microporous/macroporous carbon. In some embodiments, the method further comprises, contacting the blood with an activated carbon coated with iron. In some embodiments, the whole blood is not separated into cells and plasma. In some embodiments, the blood is separated into cells and plasma. In some embodiments, the contrary substance is phosphate, creatinine, calcium, magnesium, uremic toxins or combinations thereof. In some embodiments, the carbon has a pore size distribution showing a first large population of micropores of size <2 nm and a second large population of macropores of size 50-500 nm. In some embodiments, the carbon further comprises mesopores of size 2-50 nm. In some embodiments, the carbon is in the form of beads. In some embodiments, the carbon is in the form of a monolithic porous carbon structure. In some embodiments, the monolithic porous carbon structure has (i) continuous channels through which blood can pass with a channel size of between 200 and 1000 μm; (ii) wall thickness between 200 and 1000 μm; (iii) macropores within the walls with a mean pore size of between 1 and 50 μm; and (iv) pores within the carbon matrix suitable for the adsorption of middle and high molecular weight molecules with a mean pore size between 2 and 500 nm.

In some embodiments, disclosed is an activated carbon comprising an iron molecule bound thereto. Disclosed is an activated carbon comprising iron oxides and hydroxides. Disclosed is an activated carbon comprising iron oxides or hydroxides. Iron Oxide/hydroxide (FeO(OH)) has shown be an important sorbent of phosphates. Activated Charcoal (or Activated Carbon) by itself has very little potency to remove them, but Activated Carbon doped with Iron Oxide/hydroxide has been already studied for the removal of phosphates in wastewater. Activated carbons are a very versatile material and has been modified and doped for different purposes for decades. Due to the high surface area of activated carbons, it is possible to disperse a considerable amount of iron oxides/hydroxides nanoparticles over its surface creating an important number of binding sites for the chemisorption of phosphates on it.

Disclosed is a hemodialysis system comprising: a hemodialysis machine including, a dialyzer; and a phosfilter. In some embodiments the phosfilter is connected directly to the dialyzer.

Disclosed is a pharmaceutical composition comprising activated carbon particles bound to iron, for oral administration. The pharmaceutical composition may be for (use in) the treatment of gastrointestinal (GI) dysfunction and/or diseases or malfunction of the GI tract or the like. For example, the pharmaceutical composition may be for microbiome disorders. The pharmaceutical composition may be for the generation of uremic toxins.

Dialysate solution, also commonly referred to as dialyzing fluid, is an aqueous electrolyte solution that is similar to the found in extracellular fluid with the exception of the buffer bicarbonate and potassium. Dialysate solution is almost an isotonic solution having an osmolality of approximately 300±20 milliosmoles per liter (mOsm/L). To ensure patient safety and prevent red blood cell destruction by hemolysis or crenation, the osmolality of dialysate must be close to the osmolality of plasma which is 280±20 mOsm/L. Dialysate solution commonly contains six (6) electrolytes: sodium (Na+), potassium (K+), calcium (Ca2+), magnesium (Mg2+), chloride (Cl—), and bicarbonate. Dialysate also contains a seventh component, the nonelectrolyte glucose or dextrose. Disclosed is a dialysate solution comprising activated carbon particles bound to iron. A dialysate solution suitable for peritoneal dialysis and comprising iron containing activated carbon beads is disclosed. Disclosed is an aqueous composition suitable for peritoneal dialysis and comprising: iron containing activated carbon beads. A treatment of dialysate solution which circulates past the membrane of an artificial kidney for removal of phosphate, creatinine, calcium, magnesium, uremic toxins, indoxyl sulfate, or combinations thereof from a patient is disclosed, which solution employs activated carbon particles bound to iron. In some embodiments, the dialysate solution is used for HD and/or peritoneal dialysis. There are various types of peritoneal dialysis therapies, including continuous ambulatory peritoneal dialysis (“CAPD”), automated peritoneal dialysis (“APD”), tidal flow APD and continuous flow peritoneal dialysis (“CFPD”).

Disclosed is a method of providing hemodialysis includes the step of introducing dialysate to the hemodialysis machine. In some embodiments, the dialysate comprises activated carbon bound to iron. In some embodiments, the hemodialysis machine comprises a dialyzer and the dialyzer comprises a phosfilter. In some embodiments, the hemodialysis system is a closed loop blood flow path which transports dialysate through the dialyzer.

In some embodiments, disclosed is a composition for use in the treatment of an HD patient where a first component comprises a synthetic carbon particle containing bound iron. In some embodiments, the synthetic carbon particle mixture comprises a first carbon particle having pore size x and a second carbon particle having pore size y where y is greater than x. In some embodiments, y is two times x.

Disclosed is a method comprising contacting a bodily fluid with the composition comprising: a first component comprising a synthetic carbon particle with an iron particle bound to the surface, internally or both. In some embodiments, the contacting occurs in an extracorporeal apparatus having a first column. In some embodiments, the first component is disposed within the first column. In some embodiments, there is fluid communication between the first column and a dialyzer. In some embodiments, the bodily fluid comprises whole blood.

Disclosed is a composition for use in the treatment of HD patients, patients that are CKD (non-dialysis dependent), patients that have cancer, autoimmune disease, sepsis, AKI (acute kidney injury), inflammation, infections, intoxications, liver damage, and/or brain diseases where a first component comprises a synthetic carbon particle mixture comprising iron bound thereto.

Disclosed is a method of detoxifying a subjects blood comprising (i) contacting the plasma with a composition comprising a synthetic carbon bound to an iron.

Disclosed is a container comprising at least one filter and a micronized carbonaceous material bound to iron.

A method of treating a patient comprising contacting the patient's blood with an activated carbon bound to an iron molecule. In some embodiments, the iron particles comprise one or more compounds selected from the group consisting of Fe2O3 and Fe3O4.

### D. Ion-Selective and Metabolite Adsorbent Devices and Filtration Systems

Adsorbent device comprises an inlet and an outlet through which any suitable fluid can pass. In one embodiment, an adsorbent device can comprise a micro- or nano-particle that has been treated with an adsorbing complex. Adsorbent micro- or nano-particle can be enclosed in a device housing. Adsorbent device can comprise any suitable size or shape and orientation having inlet and outlet through which a biofluid may pass. In one embodiment, an adsorbent device can be configured for filtering blood or bodily fluids in which removing undesirable biological molecules, impurities or contaminants is desired.

The adsorbent filtration system may be generally configured for treating a biofluid, for example, bodily fluids or blood, however, any suitable fluid is contemplated. Although it is not necessary to understand the mechanism of an invention, it is believed that a fluid can be passed over, and/or through an adsorbing micro- or nano-particle comprising adsorbent complexes attached thereto for selectively and permanently binding to undesirable substances present in the fluid, for example, phosphate ions in biofluids. In one embodiment, the adsorbent complexes include, but are not limited to, ferric oxide hydroxide (FeOOH) and may contain and activated charcoal.

FIG. 8 also illustrates a hemodialysis system or circuit with addition of a hemoadsorbent device, the system generally designated for removing phosphates from blood. Currently, there are no available phosphate-selective and/or uremic toxin metabolite blood adsorptive devices on the market which can selectively remove these substances. Existing devices that provide filtration, e.g., hemodialyzers, can remove some phosphates through simple diffusion. In contrast, adsorptive devices as disclosed herein comprise a whole blood adsorptive device that selectively adsorbs phosphates or metabolites onto its surface, interior, or both through high capacity binding and can be integrated into a commercially available hemodialysis set-up or system. The level of phosphate binding capacity to the treated micro- or nano-particle can be tailored in the adsorbent device based on the ratio of adsorbent complexes to surface area. Preliminary studies of the device activated carbon-iron particles demonstrated a high specific surface area (1465.3 m²/g) and meso/macro pore volumes (1.633 cm³/g) available for binding to phosphates. In some embodiments, the specific surface area was in the range of 1000-2000 m²/g) and meso/macro pore volumes was in the range of 1.1-2.0 cm³/g). The estimated projected adsorption capacity for this technology is ˜145 mg/g. In some embodiments, estimated projected adsorption capacity for this technology is in the range of ˜120-180 mg/g. In some embodiments, Fe adsorbent will be loaded to the activated carbon beads to achieve 200-400 mg phosphate adsorption capacity. See generally FIG. 2

Referring to FIG. 8, hemodialysis circuit or system can comprise an extracorporeal blood circuit for dialysis of blood from a hemodialysis patient. An in-line hemofiltration/hemoadsorption device for hyperphosphatemic hemodialysis subjects or patients is contemplated. The system can comprise a plurality of adsorption devices positioned along a circuit for serial processing of blood, the devices being connected by one or more sets of blood tubing for continuously flowing blood through system. Using this configuration, blood may be continuously extracted from a patient's body, for example, extracted from the subject's arm into a tube to provide a continuous flow through system that can result in single-pass decontamination. Subsequent to passing through system, the decontaminated blood can then be continuously fed back into the patient's circulatory system.

System can further comprise one or more pressure monitors distributed at various points throughout the circuit for monitoring various pressures associated with blood flowing through the circuit. For example, a first pressure monitor of system can be positioned along a tube, cylinder, or cartridge proximate to where the blood is extracted from the subject's arm to measure arterial pressure. Arterial pressure can be monitored either pre-pump or post-pump depending on the type of hemodialysis machine and blood tubing being used. The pressure readings for pre-pump versus post-pump arterial monitors may provide different information regarding the hemodialysis treatment and/or the patient's access. For example, arterial vascular access and clotting problems may be identified using the first pressure monitor of system.

System can further comprise a pump for controlling fluid flow rates. Fluids (e.g., blood) flowing along a hemodialysis circuit can be treated using a heparin pump employed to inject heparin into blood leaving the body before passing through a dialyzer for preventing the blood from clotting. The hemodialysis circuit may further comprise a dialyzer including but not limited to, a coil type (FIGS. 7A and 7B), flat plate-type, laminate type, hollow fiber, a continuous hemofilter, or any type of dialyzer as known in the art which may or may not utilize a dialysate. In various embodiments, the adsorbent substance is a sheet, and the adsorbing complex is attached to a surface of the sheet. In various embodiments, the sheet is a polymer sheet. In one embodiment, dialyzer is configured for continuously flowing blood and a dialysate through the dialyzer at the same time. In one embodiment, dialysate may flow through the dialyzer from a lower passage to an upper passage. Dialysate can collect waste products, and then drain out through the upper passage to be discarded. As known in the art, dialysate and blood may flow in opposite directions in a blood dialyzer.

Notably, adsorbent devices in accordance with embodiments described herein can be positioned upstream and/or downstream of dialyzer for processing the blood either before or after the blood passes through dialyzer to undergo dialysis. In one embodiment, adsorbent devices can comprise standalone devices or may be positioned within dialysis circuitry either as a separate portion or integrally formed with dialyzer such that blood can pass through adsorbent devices simultaneously. Adsorbent device and dialyzer can be configured for removing substances or impurities from blood and/or dialysate contacting adsorbent device. In one embodiment, the adsorbent device can comprise a medical grade silicone coated with a cured polymer resin and phosphorus-selective or metabolite adsorbent finish for selectively removing phosphate from the blood. In another embodiment, the adsorbent device can comprise micro- or nano-particle and phosphorus-selective or metabolite adsorbent finish for selectively removing phosphate from the blood Once the blood passes through adsorbent device and dialyzer or from dialyzer to adsorbent device, it can then pass through an air trap and detector before being continuously fed back into the arm of a patient.

### E. Ion-Selective Treatments

In one embodiment, adsorbent device can be used to selectively filter blood to prevent and treat hyperphosphatemia or accumulation of metabolites in patients suffering from end-stage renal disease (ESRD). For example, adsorbent device could be positioned upstream and/or downstream of a dialyzer (FIG. 8) for selectively removing materials such as phosphate from the blood either before or after the blood is dialyzed.

An adsorptive complex bound to a micro- or nano-particle surface, interior, or both can be designed to selectively remove phosphate ions with little to no effect on the concentrations of other blood electrolytes or other blood chemistry compounds. See, Table 2.

TABLE 2
Chemistry Panel for Bovine Blood at Baseline and After Prosfilter Exposure

| | Na+ | K+ | Cr | BUN | Cl− | HCO3− | Albumin | Mg2+ |
|---|---|---|---|---|---|---|---|---|
| | (mEq/L) | (mEq/L) | (mEq/L) | (mEq/L) | (mEq/L) | (mEq/L) | (mEq/L) | (mEq/L) |
| Baseline | 145 | 7.7 | 0.80 | 8.5 | 96 | 32 | 3.5 | 1.7 |
| Post Phosfilter | 143 | 7.4 | 0.75 | 8.3 | 101 | 22 | 3.7 | 1.9 |

An adsorbent device can comprise a stand-alone single or multiple use in-line hemoadsorption device that provides selective blood phosphate or metabolite adsorption. It can comprise an off-the-shelf device that is easily integrated within existing hemodialysis circuitry and requires no additional treatment or monitoring equipment. (FIG. 8) An adsorbent device may comprise unique phosphate or metabolite selective adsorptivity to provide significant reduction in blood phosphate levels beyond what is offered by dialysis alone or in combination with controlled diet or drugs. In one embodiment, an adsorbent device comprises pumps that operate at typical blood flow rates prescribed during hemodialysis (i.e., for example, 200 to 500 mL/min to include pediatrics) and will pass directly back into the patient, with no additional filtration or treatment.

In one embodiment, adsorbent device can comprise a treated micro- or nano-particle and filled into a housing unit. Devices and systems described herein can comprise components including, but not limited to, a medical grade polymer and/or micro- and nano-particles comprising a plurality of phosphorus-selective or metabolite adsorbent complexes, a housing unit; and a dialyzer.

In some embodiments, adsorbent devices described herein can be easily substituted into housings similar to those used for dialyzers or hemoperfusion cylinder or cartridges and can provide rapid implementation of the presently disclosed phosphate or metabolite binding technology at a low cost. Blood can be pumped at the typical rates prescribed during hemodialysis (200 to 500 mL/min) over and/or through the adsorbent micro- or nano-particle formed into a cylinder (treated using any method described earlier to secure adsorbing complexes such as ferric oxide hydroxide), and will pass directly back into the patient.

The adsorption devices can also be incorporated into conventional hemodialysis circuitry for selectively adsorbing phosphate or metabolites during hemodialysis. In one embodiment, an adsorbent device using an adsorbing substance can be incorporated into an apparatus at least similar in physical design to flat plate dialyzers known in the art, to enable enough of the adsorbent micro- or nano-particles to be used in a device or system.

A method of filtering a contaminated biofluid includes contacting a micro- or nano-particle with an adsorbent complex. This can include adhering a cured polymer gel resin comprising ferric oxide hydroxide (FeOOH) groups to a micro- or nano-particle and may contain an activated carbon. The method can further comprise contacting a contaminated fluid with the adsorbent micro- or nano-particle. The contaminated fluid can be pumped or passed over and/or through an adsorbent micro- or nano-particle. Any suitable method of contacting a biofluid with an adsorbent micro- or nano-particle can be used. The method can further comprise adsorbing undesired particles or ions onto the adsorbent micro- or nano-particle. Undesired particles can be adsorbed onto the adsorbing complex on the surface, interior, or both of the treated micro- or nano-particle. In one embodiment, ferric oxide hydroxide (FeOOH) can be used to adsorb phosphate from blood or biofluids.

In sum, adherence (surface, interior or both) of selectively adsorbent chemistry to materials can be adapted for adsorption of non-selective or other selective agents. In some embodiments, the adsorbing complexes can be adhered to a cured polymer gel resin. In one embodiment, polymer materials that have been adhered with adsorbing particles can be used to remove phosphates from blood. In other embodiments, polymer materials with adsorbing particles can be used for treatment of sepsis/septic shock, acute liver failure, and removing metabolites, cells, toxins, drugs, antigens, and antibodies.

### F. Phosphate Filter Device

The device comprises activated carbon particles containing iron and contained within polycarbonate hemofilter housing. In some embodiments, the device comprises a single filter. In some embodiments, the device comprises additional filters. In some embodiments, the device comprises a one-five filters. In some embodiments, the device is selective for phosphate. In some embodiments, the device is selective for uremic toxins. In some embodiments, the device is selective for phosphate and uremic toxins. In some embodiments, the device is extracorporeal, e.g., outside the body. In some embodiments, the device is wearable or implantable devices for ESRD patients to take the place of traditional HD. The effective volume of the prototype device is between about 100-300 mL and this is advantageous over hemofiltration cartridges that typically have extensively larger volume requirements. Pore size distribution, PZC, bulk density and specific surface area of activated carbon particles containing iron (A13023 Fe1) have been characterized (FIG. 10). FIG. 10 shows pore structure of support carbon (A13023) and carbon containing bound iron (A13023-Fe1). The graph represents the pore size distribution of meso- and macro-pores for both samples. The table shows bulk density and surface properties of samples and differences due to iron loading: PZC=point of zero charge. In some embodiments, the iron is capable of getting in bigger pores that are on outside of the activated carbon and cannot get into smaller interior pores. Preliminary in vitro results demonstrate specific surface area for the iron containing (surface, interior or both) activated carbon particles of 1465 m²/g. In some embodiments, the particle sizes 1-10 microns. Energy Dispersive Spectroscopy (EDS) demonstrated the presence (and density) of iron on washed unused activated carbon particles (FIGS. 11A-B) and presence of both iron and phosphate on the particles after exposure to bovine blood (FIGS. 11C-D). FIGS. 12A-C show an embodiment of activated carbon A13023-Fe1: iron (FIG. 12A), oxygen (FIG. 12B) and carbon (FIG. 12C) presence primarily on the bead surface. EDS graphic (FIG. 12D) shows the weight % of each element. FIG. 12E shows an electron image of bead prior to exposure to blood. These results show modifications to the activated carbon that incorporate iron predominantly on the exterior to adsorb blood phosphate. FIGS. 13A-C show an embodiment of activated carbon A13023-Fe1: iron (FIG. 13A), oxygen (FIG. 13B) and carbon (FIG. 13C) presence in the bead interior. EDS graphic (FIG. 13D) shows the weight % of each element. FIG. 13E shows electron image interior. These results suggest the potential for more iron loading on the interior. FIGS. 14A-D show an embodiment of activated carbon A13023-Fe1 after exposure to bovine blood for 4 h at 37 C: iron (FIG. 14A), oxygen (FIG. 14B), carbon (FIG. 14C) and phosphate presence (FIG. 14D) primarily on the bead surface. Additionally, microscopy supports the presence of phosphate primarily on the bead surface in a similar pattern to iron (FIG. 14E). FIG. 14F shows electron image of the surface. These results suggest that modifications that incorporate iron on the exterior will further enhance phosphate adsorption. FIGS. 15A-D show an embodiment of activated carbon A13023-Fe1 after exposure to bovine blood for 4 h at 37 C: iron (FIG. 15A), oxygen (FIG. 15B), carbon (FIG. 15C) and phosphate presence (FIG. 15D) in the bead interior. FIG. 15E shows electron image of the interior of the bead. These results suggest that modifications that incorporate iron on the exterior will further enhance phosphate adsorption.

### EXPERIMENTAL

The following examples are provided, without limitation, for illustrating possible embodiments and embodiments of the subject matter disclosed herein. However, those of ordinary skill in the art should, in light of the present disclosure, appreciate that changes can be made in the specific examples and embodiments disclosed and still obtain a like or similar result without departing from the spirit and scope of the presently disclosed subject matter.

### Example I

### Selective Ion Removal Using a Hemodialysis Circuit

Micro- or nano-particles treated with adsorbing complexes described herein may be tested in a blood circuitry system. A typical human hemodialysis circuit may be evaluated when the addition of an adsorbent device is used. In hemodialysis circuits, arterial blood can be driven by a pump through the dialyzer and then return to the patient as venous blood.

The circuit was tested using a reservoir of bovine whole blood and passed through adsorbent devices. The blood was maintained at physiological pH (7.4) and temperature (37° C.) during the experiments and circulated by a variable flow peristaltic pump, for example, a pump manufactured by Fisher Scientific, Pittsburgh, Pa. A constant heparin infusion of 150 U/hr was maintained via an infusion pump, for example, a pump manufactured by KD Scientific, Holliston, Mass. to prevent clotting of blood in the dialyzer and phosphate hemofilter. A standard bicarbonate dialysate solution containing potassium and calcium can be prepared and pumped through the dialyzer by a variable flow peristaltic pump in a direction countercurrent to the direction of blood flow.

Blood samples were collected before and after passage through the hemoadsorption device at times 0, 30, 60, 120, 180, and 240 minutes, or any suitable time intervals. Adsorption data from the studies were calculated and presented as a time course. See, FIG. 2.

### Example II

Selective Removal of Phosphate from a Fluid

This example presents experimental data showing that an adsorbent device comprising an ferric oxide hydroxide (FeOOH) adsorbent complex selectively and efficiently removes phosphate ion (e.g., PO₄) from a passing fluid. In this experiment, a cylinder test with 50 ml heparinized bovine blood was contacted with a ferric oxide hydroxide (FeOOH)-silicone adsorptive device for approximately four (4) hours. The data show that another inorganic ion, namely iron comprised in the adsorbent, remains relatively unaffected, demonstrating limited shedding from the adsorptive device. See, Table 3.

TABLE 3
Selective Removal of Phosphate from Blood
with Limited Effects on Serum Iron

| | Phosphate | Phosphate | Iron | Iron |
|---|---|---|---|---|
| | before | After | before | After |
| | (mg/dL) | (mg/dL) | (mg/dL) | (mg/dL) |
| Control (Silicone | 7.8 | 7.8 | 129 | 142 |
| tape only | | | | |
| Fe-silicone 1 | 7.9 | 4.2 | 163 | 134 |
| Fe-silicone 2 | 7.8 | 4.2 | 129 | 119 |

Phosphate (PO₄³⁻) was also observed to be removed over time using a ferric oxide hydroxide (FeOOH). See FIGS. 3A and 3B. Phosphate (PO₄³⁻) was also observed to be removed over time using a ferric oxide hydroxide (FeOOH) attached to activated carbon nanoparticles (FIGS. 3A-B). See also FIG. 16 showing in vitro removal of phosphate by the phosfilter at different concentrations. See also FIG. 21 showing indoxyl sulfate reduction with the phosfilter. Solutions containing phosphate at 35, 45, 60, and 80 mg/L were exposed to A13023-Fe beads in a beaker at room temperature and evaluated over 2 h. Aliquots of solution were sampled and assayed for phosphate concentrations. See FIGS. 17A-C show an exemplary Phosfilter which resembles a hemofilter and contains phosphate adsorbing carbon particles in a polycarbonate housing. 17A is closed, 17B is open and 17C is a perspective view.

### Example III

Selective Ion Removal by an Adsorptive Device Coupled with a Dialyzer

This experiment was performed in accordance with Example I with the exception that a dialyzer was placed in series with a ferric oxide hydroxide (FeOOH) adsorption device. The difference between blue and orange column is the portion that removed by the prototype, demonstrating that the addition of the adsorptive device improved the phosphate removal efficiency as opposed to dialysis alone. Further, analysis also showed little or no changes in iron concentration over the same time period thus showing that the ferric oxide hydroxide (FeOOH) adsorptive device demonstrated limited shedding of ferric substrate.

FIGS. 3A-B presents exemplary data demonstrating the phosphate reduction with (3B) and without (3A) a FeOOH prototype. FIG. 4A presents exemplary data demonstrating the phosphate reduction by ferric oxide hydroxide adsorption complexes with three distinct activated charcoal chemistries during in vitro blood studies conducted over 300 minutes. The starting phosphate concentrations were 60-70 mg/L, with attainment of normal and sub-normal levels achieved with two of the three chemistries.

FIG. 4B presents exemplary data demonstrating the phosphate reduction.

Functionalized activated carbon with iron oxides and hydroxides were developed. For example, eight different strategies can be applied to functionalize Iron Oxide/Hydroxide Activated Carbons:

1—Add an Iron salt that is soluble in the solvent that is used to prepare the activated carbon polymer precursor, then Carbonize/Activated the precursor to over 1000 C to obtain a Carbon that has stable Iron oxide particles on its surface.

2—To an already synthesized Activated Carbon, add an Iron salt and thermally treat it to 700 C to create stable Iron Oxide particles on the carbon surface.

3—Impregnate activated carbon with an Iron (III) salt followed by a chemical stabilization promoting precipitation of Iron Oxide/Hydroxide on Activated Carbons surface. The carbon is washed several times to remove loose Iron oxide/hydroxide particles.

4—Similar to 3 but following a more specific method (Incipient Wetness Impregnation) were a saturated solution of the given salt (FeCl3*6H2O) is put in contact with the minimum amount of Carbon that can absorb that fluid, not leaving any aliquot, obtaining the maximum load of Iron possible on carbon.

5—Similar to 3, but using, as in 4, a saturated solution of Iron (III) salt.

6—Similar to 5, but using half saturated solution of Iron (III) salt.

7—Similar to 5, but drying the iron salt in rotavap previous to neutralization.

8—Similar to 7, but doing a neutralization in milder conditions.

Table 4 shows the sorbents used for this study and the corresponding bulk density. Carbons 1 to 6 were reported in previous report (November 2022) being Carbons 7 and 8 the ones subjected to comparison. It shows that FeO(OH) is several times heavier than any of the carbon derived sorbents. The carbon sorbents are numbered following the different strategies to obtain them. It is noticeable that Carbon 7 is much heavier than Carbon 8 that indicates a higher load of Iron. That important difference is due to the milder process of neutralization in Carbon 8 compared to 7.

TABLE 4
Sorbents Used and its corresponding Bulk Density:

| | sample | | Bulk Density (g/ml) |
|---|---|---|---|
| FeO(OH) | | | 1.180 |
| | 1. | RDA18122 | 0.339 |
| | 2. | A194-Fe700 | 0.225 |
| | 3. | RA19922-Fe | 0.208 |
| | 4. | RA19922-Fe4 | 0.287 |
| | 5. | A) RA19922-Fe3 | 0.273 |
| | | B) RA19422-Fe1 | 0.203 |
| | 6. | RA19422-Fe2 | 0.198 |
| | 7. | RA32622-Fe4 | 0.273 |
| | 8. | RA13023-Fe1 | 0.219 |

Next, Iron containing Carbons were evaluated for microparticulate. Samples (carbons 6, 7 and 8) were analyzed with AccuSizer PSS machine. Table 5 also shows results for materials in Table 1 above.

TABLE 5
Particle count (in units/ml)

| | Limit | RA19422- | RA19422- | | |
|---|---|---|---|---|---|
| | (#/ | Fe2 | Fe2 | RA32622- | RA13023- |
| Samples | mL) | (I) | (II) | Fe4 | Fe1 |
| Particles >10 μm | 25 | 34.0 | 40.5 | 1 | 2 |
| Particles >25 μm | 3 | 6.0 | 12.3 | 0 | 0 |
| Pass (Y/N) | N | N | N | Y | Y |

TABLE 6
Particle count (in units/ml)

| Samples | FeO(OH) | RA19422-Fe2 (I) | RA19422-Fe2 (I) |
|---|---|---|---|
| Particles >1 μm | 19700 | 1850 | 1710 |
| Particles >5 μm | 1380 | 1120 | 574 |
| Particles >10 μm | 0 | 34.0 | 40.5 |
| Particles >15 μm | 0 | 10.0 | 23.9 |
| Particles >25 μm | 0 | 6.0 | 12.3 |
| Particles >50 μm | 0 | 3.0 | 1.23 |
| Pass (Y/N) | N | N | N |

Next, in vitro efficacy testing/phosphate removal experiments were performed.

Preparation of Solution with 75 mg/L:

### Reagents:

Isolate multi-Electrolite Injection pH: 7.4 (Braun): Contains Ca(2+), Mg(2+). Acetate, Cl(−), Gluconate and HPO4(2−) (0.5 mmol/L).
Phosphate Buffered saline tablets (Fisher Bioreagents)

1 tablet of Phosphate Buffered Saline tablets were diluted in 1000 ml of Isolate solution giving a Phosphorous concentration between 70-80 mg/L.

### In Vitro Phosphate Removal Testing:

15 ml centrifuge tubes are loaded with 0.66 ml of sorbent (obtained by weight and considering the bulk density of any given sorbent) and put in contact with 11 ml of Solution 2. 11:0.66 is equivalent to 50:3 that corresponds to 5 L of blood in an average adult human using a 300 ml column. Every tube corresponds to a given time (15, 30, 60, 120 minutes) where the tubes are gently agitated. After being removed from agitation at the given time, is centrifuged at 2000 rpm and 1 ml of supernatant is obtained for phosphate analysis. One tube without any sorbent is agitated to the end of the experiment as Control and compared with a sample of Solution 2 to validate the experiment.

### Colorimetry Analysis of Phosphates in Spiked Isolate Solution:

Abcam ab65622 Phosphate Assay Kit (colorimetric) was used to measure the phosphate concentrations in initial solution and in phosphate removal experiments.

Abcam ab65622 Phosphate Assay Kit (colorimetric) was used to measure the phosphate concentrations. FIG. 4B shows the results of phosphate adsorption for FeO(OH) and carbons 7 (a32622-Fe4) and 8 (A13023-Fe1). FeO(OH) removes most of phosphates very quickly, depleting the solution from it. Both carbons showed some removal, being Carbon 8 able to bring phosphate levels into desired window of 35-45 mg/L after 30 min. Carbon 7 is much heavier and contains a higher load of Iron than Carbon 8, but it removes less phosphorous. Due to milder conditions of neutralizing during the preparation of Carbon 8, is very likely that the FeO(OH) particles formed would be smaller than in Carbon 7.

When Carbon 8 (A13023-Fe1) is washed there is a reduction of efficacy. It is very likely that the washing method removed some Iron particles on the external surface of the carbon.

FIG. 4C shows the results of phosphate adsorption for FeO(OH) and carbons 1-4 and 5A. As it can be seen, FeO(OH) removes most of phosphates very quick, depleting the solution from it. Carbon 1 and 2 showed no affinity, but both Carbons 3 and 4 showed some removal, being Carbon 4 able to bring phosphate levels into de desired window of 35-45 mg/L after 2 h. On the other hand, Carbon 5. A showed to be very effective, similar than FeO(OH).

Both Carbon 4 and 5.A where washed to remove particles. It was observed that Carbon 4 had a lot of iron particles in its surface and was continuously releasing particles when washing and that was not happening on Carbon 5.A who showed a much cleaner surface. FIG. 4D shows the removal of Carbon 5.A washed (Carbon 5A) (II)) and compared with the original. It can be observed that washing had no effect on the affinity of Carbon 5.A to Phosphates.

FIG. 4E shows a comparison of all the carbons made following method 3 and its variations (5 and 6). For samples Carbon 5.B and Carbon 6 it was not taken the value at 15 min. Reproducibility is shown when applying same method to a different batch of carbon (5.A and 5.B) had very little effect on affinity to phosphates, being minimal.

When the concentration of the iron salt solution is the half of saturation (Carbon 6), the carbon showed a very interesting behavior. Being initially almost as fast as Carbon 5.B) but saturating and staying in the targeted zone. Comparing Carbons 3, Carbon 6 and Carbon 5.B shows that how much phosphates to remove can be controlled by just changing the initial concentration that the Iron (III) salt is impregnated with the carbon.

### Example IV

Experimental data using phosphate solutions containing the iron containing activated carbon particles indicated significant efficacy in reducing phosphates, with higher adsorption capacities at higher phosphate concentrations. See FIG. 16. The device had a loading capacity of adsorbent to substrate of ˜726 g/m². In vitro studies using the device described herein and conducted during simulated HD (using bovine blood) have demonstrated mean adsorption capacities of ˜1800 mg/m².

### Example V

### Ex Vivo and In Vivo Data

The device was subjected to a 4 h ex vivo experiment employing bovine blood and demonstrated limited presence of clotting as demonstrated by the relatively clean interior after use. See FIGS. 19A-D. The activated carbon beads show integrity and without the presence of interspersed clots (FIG. 19A). FIGS. 19B and C represent the top end of the device, demonstrating limited clotting and contact with the housing. FIG. 19D shows the bottom of the device with a filter and lack of presence of blood clots.

Additionally, pressure determinations in the extracorporeal circuit were maintained within a tight range of 4.1 and 4.3 mm Hg throughout the duration of the study. In some embodiments, this range could be about 4.0-4.5 mm. Preliminary data also demonstrated reasonable efficacy, selectivity, and safety in the ex vivo studies employing bovine blood as demonstrated in chemistry and CBC assessments (FIGS. 18A-B). A mitigation strategy was subsequently employed whereby the Phosfilter was stored prior to use in a solution of calcium to bind non-specific pores on the carbon particles. A subsequent 4h benchtop study conducted in a phosphate solution showed mitigation of reductions in calcium and magnesium, while retaining reductions in phosphate.

The Phosfilter device was studied during a 4 h ex vivo experiment with bovine blood and demonstrated efficacy and safety (FIGS. 18A, 18B and 20). While slight increases in serum iron were demonstrated, a slow, physiologic delivery, may actually be advantageous in HD patients, who have a >50% prevalence of iron deficiency. Reduction in creatinine was a surprising finding, which, if present in whole animal and human studies, may serve to augment adequacy of HD. Calcium and magnesium reductions were subsequently mitigated (FIG. 18A) through saturation of the binding sites through storage of the device in a calcium solution, with a rinsing protocol employed prior to use.

### Example VI

Experimental Design and Methods: to test iron-containing activated carbon beads and Phosfilter prototypes in buffers and blood for phosphate adsorption performance and safety to select the top performing device to move forward in development.

Hypothesis: Iron-containing activated carbon beads will enhance phosphate removal from buffers and blood and will have minimal impacts on other blood components.

Benchmark: Achievement of ≥30% reduction in mass of phosphate removed during static and dynamic experiments and minimal or mitigatable alterations in hemo- and bio-compatibility tests.

Creation of Activated Carbon Beads Containing Iron. Modifications of the prototype (A13023-Fe1) bead were created for testing phosphate adsorption performance and safety in benchtop and ex vivo blood studies.

Iron will be bound to synthetic activated carbon beads using different load percentages and strategies. These methods will utilize 1) Incipient wet impregnation of iron salt precursor and further neutralization, 2) Incipient wet impregnation of iron oxide hydroxide nanobeads, and 3) Ion-exchange adsorption of iron salt precursor and further neutralization. For the first method, a known concentration of iron salt solution is added to the activated carbon. Previous studies have determined the conditions. Thus, the exact amount of precursor that is loaded inside the sorbent pores can be calculated. The samples are subsequently treated by neutralization using NaOH to transform the salt precursor into the desired iron species, such as FeO(OH), on the activated carbon surface. For the second method, instead of a salt precursor, pre-synthesized FeO(OH) nanobeads suspended in solution will be used. These beads will be in contact with the sorbent, dried, and neutralized with NaOH. This method has the capacity to produce more dispersed FeO(OH) beads of well-known particle sizes. The third method will consist of oxidizing the activated carbon surface to produce surface carboxylic groups that bind to iron salts electrostatically Fe(3+). The sample will then be treated with NaOH to produce highly dispersed FeO(OH) on the surface of the sorbent. In order to produce several new and optimized synthetic iron-containing activated carbon beads, different concentrations of iron salts and FeO(OH) will be used in the first and second methods, whereas different degrees of oxidation of activated carbon will be used in the third method. See FIG. 10. As such, symmetrical particles with meso- and macro-pore volumes and high surface area are created (FIGS. 11A-D).

Durability of Iron Binding and Bead Characterization. Following the synthesis of iron-containing activated carbon beads, durability of iron binding will be assessed in a representative sample of each bead. The beads will be subjected to thorough washing in DI water with agitation in a shaker bath for about 24 h (or about 5-36 hrs). The beads will be removed, water will be filtered, and both the filtrate and washed beads will be assayed for iron (ab83366; Colorimetric Iron Assay Kit, Abcam, Cambridge, UK) and read at 593 nm. Bead characterization will include bulk density, PZC, surface area, micro/meso/macro pore volumes and mean pore size (Table 1). These assessments will be performed (AccuSizer 780 Optimcal Particle Sizer, Poremaster 60, Tristar II Plus, and plate reader). The beads will also be observed under SEM and EDS to assess activated carbon surface and interior and loading of iron. Microscopy will be performed using a Thermo Fisher Scientific Helios 5-CX focused ion beam scanning electron microscope (FIB-SEM).

Phosphate Adsorption Studies from Iron-Containing Carbon Beads. Characterization of the adsorptivity of the beads will confirm the necessary device size/surface area for adsorption. The prototyped beads (A13023 and A13023-Fe1) will serve as negative and positive controls. For static experiments, an aqueous solution (50 mL) containing phosphate (3-12 mg/dL) and BSA (about 40 g/L or 20-50 g/L) will be studied. Different iron containing beads (n=10) will be studied separately by adding 0.7-0.8 g (depending on bulk density of carbon) (or about 0.5-1.0 g) to each aqueous solution at 37 C and pH 7.4 in a shaker bath. Samples will be obtained at 15, 60, 90, 120, 180, 270, and 300 min. after adding beads. Experiments will be conducted in triplicate. These studies will focus on efficacy as defined by mass of phosphate removed and lowering of phosphate concentrations over the experiments. The phosphate adsorption studies will be conducted at Katharos' start-up laboratory space (see Letter) using a commercial assay (ab65622; Colorimetric Phosphate Assay kit, Abcam, Cambridge, UK) and plate reader at 650 nm.

Phosphate Adsorption and Safety Studies from Phosfilter Prototypes. A series of ex vivo studies will be conducted to study the adsorbing properties of Phosfilter prototypes and to derive preliminary assessments of hemocompatibility and biocompatibility prior to animal studies. The top 2-4 performing beads (from static experiments) will be prioritized. All ex vivo studies will be performed using a dedicated hemoperfusion platform (Aimalojic.com) incorporating a prototype Phosfilter. These dynamic experiments will be conducted in fresh bovine blood (1.6 mL or about 1-2 ml) supplemented with neutral phosphate solution to achieve phosphate concentrations of 3 to 12 mg/dL. Hemoperfusion sessions will be conducted at blood flow rates (Qb) of about 100-150 mL/min. Instantaneous clearance, extraction ratio, mass removal, and saturation of the Phosfilter will be determined at 15, 60, 90, 120, 180, 270, and 300 min. from the inlet and outlet ports of the Phosfilter. Blood centrifugation (about 10 min., 3000 rpm) will occur within 1 h of collection. All experiments will be conducted at 37 C in triplicate. Efficacy assessments will focus on primary (mass of phosphate removal over the experimental duration) and secondary (creatinine and urea removal) outcomes. Safety assessments will include changes in hemocompatibility (CBC) and biocompatibility (urea nitrogen, creatinine, SDMA, calcium, albumin, total protein, sodium, potassium, chloride, glucose, bicarbonate, CRP, ferritin, transferrin, iron). All blood studies will be assessed on an IDEXX in the Cowgill laboratory. Risk Mitigation. The current prototype has sufficient performance to move forward into canine studies; de-risking reliance of Aim 2 on Aim 1. As calcium and magnesium concentrations were reduced in the initial blood studies (see preliminary data), we have tested and incorporated calcium into our storage conditions.

Since calcium and magnesium concentrations were reduced in the initial blood studies, calcium will be incorporated into the storage solutions for the activated carbon-iron particles and thoroughly rinsing prior to use. The solution per product will be optimized to prevent reductions in calcium and also prevent potential release of calcium during use.

### Example VII

Specific Aim 2: Test the Phosfilter in a canine clinical model of kidney disease requiring hemodialysis. In this aim, efficacy, safety, hemocompatibility, biocompatibility, and phosphate removal kinetics of the Phosfilter in 6 dogs with kidney disease requiring HD will be evaluated. The application of a novel phosphate hemoadsorptive device, the Phosfilter, will promote in vivo phosphate clearance compared to a hemodialyzer alone in canine patients undergoing HD for kidney failure.

Benchmark: Additional reduction in serum phosphate concentrations of 1 mg/dL in HD plus Phosfilter combination vs. HID treatment alone.

Animal Model. Dogs (n=6; male and female; sex as a biological variable) presented to the William R. Prichard Veterinary Medical Teaching Hospital for the dialytic management of acute or chronic kidney failure will be evaluated. All study procedures and assessment will conform to standard of care for IRIS Grade or Stage specific acute kidney injury (AKI) or chronic kidney disease, respectively and approved by IACUC at the University of California, Davis. Animal owners will provide consent. The canine studies will be conducted under Dr. Cowgill given his expertise in kidney diseases and extracorporeal device 46-53

The following two groups of dogs will be recruited for entry into the study: 1) Incident dogs with AKI and 2) Dogs initiating or undergoing chronic maintenance HD. Canines will be enrolled to receive up to 4 intermittent HD/phosphate hemoadsorption (IHD/PHA) treatments. Other Inclusion Criteria: 1) age 1-15 y, 2) weight about >15 kg, 3) hemoglobin □9 g/dL, and 4) IHD duration of 5 h. Exclusion Criteria: 1) PCV about ≤21%, 2) requirement for CRRT procedures or dialysate-based procedures, 3) requirement for phosphate additives to dialysate, 4) pregnant or lactating, 5) history of hypocalcemia or hypophosphatemia, 6) history of allergy or sensitivity to extracorporeal membranes or components of the Phosfilter.

An initial evaluation will include staging for CKD or grading for AKI. Prior to extracorporeal therapies, urinary clearance of creatinine, urea, and phosphate will be determined to establish GFR, and residual urea and phosphate clearance. Urinary clearances will be measured from a quantitated about 1 h urine collection via indwelling catheter. Plasma creatinine, phosphate, and urea will be obtained at the midpoint (about 30 min.) of collection.

Control Treatment. Each dog will receive up to 3 HD sessions utilizing a high flux hemodialyzer (F160NR, Fresenius) (FIG. 6). The dialysis dose for subjects with AKI must be delivered with consideration of the degree of azotemia to prevent dialysis disequilibrium. Enrollment of subjects will be deferred until pre-dialysis BUN has been controlled by HD sessions to about ≤200 mg/kg. Each study will be configured to deliver a Kt/V dose of 1.2 during the 5 h study. For animals requiring or tolerating a larger treatment dose, the session will be extended to deliver an effective treatment (typically a Kt/V between 2.5 to 3.5 for maintenance). The clinical, biochemical, and coagulation profile of the animals will be measured.

Experimental Treatment. Each dog will receive up to 4 HD sessions (about 5-h treatment) utilizing the Phosfilter in combination with high flux hemodialyzer at the same dialysis dose as in Control Treatment and same blood profile assessments (FIG. 6). Control and Experimental Treatments will be randomized when possible.

The Extracorporeal Protocol. All procedures will be performed on a Gambro Phoenix HD system. All procedures will use heparin anticoagulation and ACT monitoring (Vertebrate Animals). Dialysate flow will be 500 mL/min and ultrafiltration will be provided as required. The Phosfilter will be placed upstream of the hemodialyzer in the extracorporeal circuit (FIG. 8). Acute Safety Assessments. Assessments (hematocrit, O2 saturation, non-invasive blood pressure, heart rate, respiratory rate, rectal temperature) will be recorded at about 0, 15, 30, 45, 60, 90, 120, 150, 180, 210, 240, and 300 min. during each session. If pre-treatment phosphate is <5.0 mg/dL, the treatment session will not employ the Phosfilter. Monitoring and treatment for signs of hypocalcemia and hypophosphatemia are described (Vertebrate Animals).

Hemocompatibility. Platelets: Platelet count, morphology, and distributed platelet volume will be determined as predictors of platelet activation within the Phosfilter and control dialyzer. Visual inspection for clotting will be conducted using a semi-quantitative visual scale. Clotting in the air trap and blood line will be scored as: 1=no clotting; 2=presence of fibrinous ring; 3=clot formation (up to 5 cm), 4=coagulated system. Scoring for clotting in the dialyzer and Phosfilter will be: 1=clean dialyzer/Phosfilter; 2=a few blood streaks (affecting less than about 5% of the surface); 3=any blood streaks (more than about 5%), 4=coagulated dialyzer/Phosfilter. Platelet count will be assessed prior to the start of the procedure and from the common inlet port and simultaneously from the outlet ports of the Phosfilter and hemodialyzer (alone or in combination) at 30, 60, and 300 min. White Blood Cells (WBCs) and Red Blood Cell (RBC) indices: These will be assessed as an index of hemocompatibility of the Phosfilter and control dialyzer (alone or in combination). They will be measured as described above. All hematologic assessments will be performed in the Clinical Chemistries Lab of UC Davis Veterinary Medicine.

Biocompatibility. Blood will be collected from the catheter prior to the start of each session and from the inlet (arterial) port at the end of the session (300 min.) for BUN, creatinine, SDMA, calcium, albumin, total protein, sodium, potassium, chloride, glucose, bicarbonate, and C-reactive protein. Assessments will be performed on IDEXX Vet Station or Catalyst One Instrumentation. Additional testing to Texas A&M Veterinary Diagnostic Laboratory will include ferritin, transferrin, iron, iPTH, FGF-23, vitamin D, d-dimer, AT3, hemolysis quantification, and markers of oxidative stress (malondialdehyde, oxidized-LDL).

Serum Phosphate, Creatinine, and Urea Kinetics. Blood will be collected from the inlet (arterial) port of the extracorporeal circuit at about 0, 15, 30, 60, 120, 180, 240, 270 min. for serum phosphate, urea, and creatinine. The instantaneous blood clearance, extraction ratio, and mass removal by the Phosfilter will be determined at about 15, 60, 90, 120, 180, and 270 min. by collection of blood from the inlet and outlet ports of the Phosfilter. For dialyzer clearances (blood urea clearance (Kdurea), creatinine clearance (Kdcreat) and phosphate clearance (KdPO4)), blood will be collected from the inlet and outlet ports of the hemodialyzer at 30 and 270 min. Ionic dialysance will be performed every 30 min. on the Phoenix platform to estimate changes in Kdurea between direct measurements. Extracorporeal blood flow, hemodialyzer fiber bundle volume, blood volume of the Phosfilter, transit time through the Phosfilter and hemodialyzer, and access recirculation will be measured at about 15, 60, 90, 120, 180, and 270 min. using a Transonics Systems HD01 blood flow monitor and Transonics Systems Fiber Bundle Volume software. Samples will also be collected at about 1 and 2 h post-treatment to assess rebound.

The complete dialysate volume (at about 500 ml/min flow) will be collected on ice (about 0-60, 60-120, 120-180, 180-240, 240-300 min.), weighed, and an aliquot collected. In addition, a continuous partial dialysate collection at a flow of about 1.0 mL/min will be collected on ice throughout the procedure. The phosphate, creatinine, and urea concentrations of each hourly aliquot of the complete dialysate volume will be measured to determine hourly excretion rate and total phosphate, creatinine, and urea mass removal due to the hemodialyzer. The phosphate, creatinine, and urea concentration of the partial dialysate collection will be measured to provide confirmatory assessment of the phosphate, creatinine, and urea removal by the dialytic component of the treatment.

Phosphate, Creatinine, and Urea Removal Efficacy Assessments. The instantaneous whole blood extraction ratio (ER), clearance, and mass removal by the Phosfilter for phosphate, urea, and creatinine will be determined from the change in respective concentrations across the device and the measured blood flow rate. The degree of saturation of the Phosfilter over time will be plotted. The plasma phosphate (and creatinine and urea) elimination rate and clearance over the treatment will be calculated independently from the sequential changes in plasma concentrations throughout the session using pharmacokinetic software and compared to the clearance and kinetic determinations of the extracorporeal circuit and dialysate. The total phosphate mass removal by the hemodialyzer will be determined from the sum of the mass removal determinations of the hourly dialysate collections and the continuous partial dialysate collection and compared to the similar determinations of the mass removal for creatinine and urea derived from urea kinetic assessments. The total mass removal for phosphate for the hemodialyzer, the Phosfilter, and the combined extracorporeal devices will be calculated using the respective time-averaged clearances of the respective hemodialyzer, and the Phosfilter, and both devices in series. Phosphate kinetics will be compared between treatments with and without an inline Phosfilter using published models as a starting point 54-56. Nonlinear regression analyses using the Phoenix® Platform (Certara, Inc.) will be used to explore multi-compartment models that best fit the study data.

Study Efficacy and Safety Endpoints, Sample Size and Statistical Analysis Plan (Both Aims). The primary efficacy endpoints are: 1) Absolute blood phosphate concentration reduction, 2) Amount (mass) of phosphate removed, 3) Phosphate clearance, 4) Phosphate reduction ratio and percentage, 5) Phosphate levels at the end of the 2 week study or time of last treatment (in cases of death), and 6) Phosfilter saturation. Secondary endpoints include phosphate levels immediately prior to the next treatment (assessment of body store reduction), phosphate kinetics, safety, tolerability, hemocompatibility, and biocompatibility between Experimental and Control treatments.

The sample size (n=6) was based on an expected change (in standard deviation) for phosphorus levels between Control and Experimental treatment of 0.6 mg/dL27. Given the 6 dogs each receiving conventional HD with and without the Phosfilter, a 0.99 mg/dL difference in serum phosphorus concentration between treatments can be detected with 90% power at α=0.05. This is clinically feasible given that conventional HD can result in a serum phosphorus change of about 2-3 mg/dL from beginning to end of treatment and it is expected that the Phosfilter will result in additional reductions. Other Primary and Secondary endpoints will be compared between treatments. Data will also be analyzed for the influence of covariates (sex, dietary phosphate intake, oral binder usage, oral intestinal transport inhibitor, residual kidney function (GFR), flow rates, acute vs. chronic kidney disease) on primary and secondary outcomes. All data will be graphed to determine a normal Gaussian distribution and data will be linearized by log transformations if required. For continuous variables, use of parametric vs. nonparametric tests will depend on whether data follows a normal distribution. Paired T-test or Wilcoxon Rank Sum test (nonparametric) will be used to compare differences between the two treatments. Discrete variables will be assessed by either Chi square or Fisher's test. Descriptive statistics will include mean, median, SD, SE, and CI.

### Expected Results, Potential Problems and Alternative Solutions.

Aim 1. The best-performing beads will likely achieve ≥30% reduction in phosphate and have minimal impacts on other blood components. Discrete changes to blood components and off-target binding may be seen. Proposed modifications could produce a carbon bead with enhanced absorptivity and safety, with safety being a high priority for to advance into canine studies. Each bead will be tested for removal of prototypical uremic toxins and durability of iron binding. The size of the Phosfilter housing and mass of beads can be adjusted.

Aim 2: The Phosfilter will promote in vivo phosphate clearance vs. hemodialyzer alone in HD. A higher phosphate load will be removed in the same timeframe. Future human studies will be scaled to blood flow rates of about 400 mL/min and 70 kg weights. Safety issues and interventions will be evaluated with the investigators and consultants. Interventions for hypophosphatemia and hypocalcemia will be performed. Hypocalcemia can be managed by high calcium (3.0 mEq/L) dialysate baths. Diet and phosphate binder usage will be recorded in the proposed study to account for changes in blood phosphates. Patient preference and quality of life assessments will be implemented in future human studies as similar tools are not currently validated in canines.

Benchmarks: activated carbon containing iron beads can: 1) achieve about ≥30% reduction in mass of phosphate removed in benchtop experiments, 2) demonstrated minimal or mitigatable alterations in hemo- and bio-compatibility, and 3) demonstrated an additional 1 mg/dL reduction in serum phosphate in canines receiving HD plus Phosfilter.

Tolerability/Hemocompatibility. Platelet count, distributed platelet volume, and platelet morphology will be determined as predictors of platelet activation within the Phosfilter and control dialyzer. Visual inspection for clotting will be conducted using a semi-quantitative visual scale. Clotting in the air trap and blood line will be scored as follows: 1=no clotting; 2=presence of fibrinous ring; 3=clot formation (up to about 5 cm), and 4=coagulated system. Scoring for clotting in the dialyzer or phosfilter will be as follows: 1=clean dialyzer/Phosfilter; 2=a few blood streaks (affecting less than about 5% of the surface); 3=any blood streaks (more than about 5% or about), and 4=coagulated dialyzer/Phosfilter. WBC count will be assessed on the Phosfilter and control dialyzer (alone or in combination). Tests for platelets and WBC will be measured on blood collected prior to the procedure and from the common arterial port at about 30, 60, 120, 180, and 240 min.

Biocompatibility. Blood will be collected from the catheter prior to the start of each session and from the arterial port at the end of the session (about 240 min.) for blood urea nitrogen, creatinine, calcium, albumin, total protein, sodium, potassium, chloride, glucose, bicarbonate, CRP, ferritin, transferrin, iron, d-dimer, AT3, hemolysis quantification, and markers of oxidative stress (malondialdehyde, oxidized-LDL). In some embodiments, PTH, FGF-23, vitD levels will also be assessed.

Potential Problems and Alternative Solutions. If the in vivo performance of the device is lower than expected and/or if any flow-related issues occur in the first canine, the back-up device will be tested. ISE the Phosfilter will be positioned to more rapidly reach a phosphate nadir. Data has demonstrated that phosphate lowering during HD (diffusion) reaches a nadir around 90 min., rebounds somewhat during the remaining session, and then rebounds just after HD to about 40% pre-dialysis levels. Specifically, fresh bovine blood (about 1.5 L) was pumped through a circuit containing the iron-containing activated carbon particle Phosfilter for 4 h at 37 C. Samples (6 mL) were obtained pre- and post-device, centrifuged and serum assayed for phosphate concentrations. Average percent reduction was about 44% during 60 min. The nadir concentration was maintained throughout the remaining 3 h of the experiment. The data demonstrated a 4.9 mg/dL reduction in serum phosphate concentration during the 4 h ex vivo study. See FIG. 20.

In some embodiments, a nadir could be reached earlier (at about 30 min.) with incorporation of the Phosfilter. It is anticipated that the addition of adsorption to diffusion will shift the curve during HD such that a higher phosphate load will be removed in the same 4 h timeframe. Additional in vivo studies employing healthy canines with induced hyperphosphatemia can also be conducted. Phosphate kinetic analysis will be included to more fully understand how the Phosfilter may impact removal, plasma concentrations, and body content of phosphate. The planned assessments are based on publications that employ a simplified physiologic approach but can be expanded to a full physiologically-based kinetic model that includes organ blood flows and organ percentages of whole body composition of phosphates (REF vitD work). This data can be used for predictions on how the Phosfilter will impact phosphate plasma concentrations and body burden in subsequentially planned human studies. The current study is anticipated to last 2 wks.

The results will accelerate the development of a device for humans that is capable of selectively adsorbing phosphates and dramatically altering the paradigm of hyperphosphatemia management in ESRD. The current study represents the final pre-clinical development of the Phosfilter and closely mirrors the intended FDA approved use of the Phosfilter in humans.

Embodiments of the present disclosure shown in the drawings and described above are examples of numerous embodiments that can be made within the scope of the appended claims. It is contemplated that the configurations of adsorption devices, systems, and methods can comprise numerous configurations other than those specifically disclosed herein.

What is claimed is:

1. A device comprising an adsorbent substance comprising at least one inorganic ion-selective adsorbing complex and/or metabolite.

2. The device of claim 1, wherein the adsorbent substance comprises a micro- or nano-particle.

3. The device of claim 1, wherein the adsorbent substance is incorporated into a cartridge.

4. The device of claim 2, wherein the micro- or nano-particle is an activated carbon particle.

5. The device of claim 4, wherein said at least one inorganic ion-selective adsorbing complex comprises a ferric oxide hydroxide (FeOOH) group.

6. The device of claim 5, wherein said ferric oxide hydroxide (FeOOH) group selectively adsorbs an inorganic ion consisting of phosphate.

7. The device of claim 1, wherein said at least one inorganic ion-selective adsorbing complex comprises a ferric oxide hydroxide (FeOOH) group, wherein said at least one inorganic ion-selective absorbing complex adsorbs metabolites.

8. The device of claim 6, wherein said adsorption device comprises a hemoadsorption device, wherein said device is configured in fluidic communication with an extracorporeal blood circuit.

9. The device of claim 2, wherein the micro- or nano-particle is an iron-based adsorbent.

10. The device of claim 9, wherein said device is a hemodialysis machine.

11. The device of claim 8, wherein hemoadsorption device further comprises a dialyzer, a housing device similar to a dialyzer, or a hemoperfusion cylinder or cartridge.

12. A method, comprising:
a) providing a device comprising an adsorbent substance comprising at least one inorganic ion-selective adsorbing complex and/or metabolite; and
b) adhering an inorganic ion-selective adsorbing complex to said adsorbent substance.

13. (canceled)

14. (canceled)

15. The method of claim 12, wherein adhering said inorganic ion-selective adsorbing complex comprises the steps of:
a) obtaining an activated carbon particle; and
b) loading said activated carbon particle with said at least one inorganic ion-selective or metabolite adsorbing complex.

16. The method of claim 15, wherein the method further comprises loading the said activated carbon particle with said at least one inorganic ion-selective or metabolite adsorbing complex into a cartridge and attaching the cartridge to a dialyzer.

17. (canceled)

18. (canceled)

19. (canceled)

20. A method, comprising:
a) providing;
i) a filtration device comprising an adsorbent substance comprising at least one inorganic ion-selective or metabolite adsorbing complex; and
ii) a biofluid comprising at least one inorganic ion or metabolite;
b) contacting said filtration device to a patient;
c) contacting said biofluid with said adsorption device whereby said at least one inorganic ion or metabolite is selectively removed from said biofluid.

21. The method of claim 20, wherein said at least one inorganic ion is selected from the group comprising or consisting of phosphate, creatinine, calcium, magnesium, uremic toxins or combinations thereof.

22. (canceled)

23. (canceled)

24. (canceled)

25. The method of claim 20, wherein said at least one inorganic ion is selectively removed from said fluid without substantially affecting the concentration of other blood chemistry compounds.

26. (canceled)

27. (canceled)

28. (canceled)

29. (canceled)

30. (canceled)

31. The method of claim 12, further comprising:
a) providing;
i) the device comprising an adsorbent substance adhered to at least one inorganic ion-selective or metabolite adsorption complex;
ii) a container comprising a first fluid at a first inorganic ion or metabolite concentration;
iii) an extracorporeal filtration circuit in fluidic communication with said container; and
b) positioning said adsorbing device within said extracorporeal circuit;
c) contacting said fluid with said adsorbing device
d) selectively removing said inorganic ion from said first fluid to create a second fluid at a second inorganic ion or metabolite concentration.

32. The method of claim 31, wherein said first inorganic ion or metabolite concentration is higher than said second inorganic ion or metabolite concentration.

33. (canceled)

34. (canceled)

35. (canceled)

36. The method of claim 31, further comprising storing the adsorbing device prior to step a in a solution of calcium to bind non-specific pores on the carbon particles.

37. (canceled)

38. (canceled)

39. (canceled)

40. (canceled)

41. (canceled)

42. (canceled)

43. (canceled)

44. (canceled)

45. The device of claim 4, wherein said activated carbon particle adsorbs an inorganic ion consisting of phosphate and or a metabolite.
